SEQUENCE LISTING (10 sequences)

SEQ ID NO: 1 - RNA, 19 nt, synthetic construct
  source 1..19: mol_type = other RNA
  modified_base order(1,3,5,7,9,11,13,15,17,19): mod_base = OTHER; note = 2-Prime-O-Methyl RNA nucleotides
  modified_base order(2,4,6,8,10,12,14,16,18): mod_base = OTHER; note = 2-Prime-deoxy-2-prime-F RNA nucleotides
  modified_base order(1..3,17..19): mod_base = OTHER; note = Phosphorothioate
tcgaagtattccgcgtacg

SEQ ID NO: 2 - RNA, 19 nt, synthetic construct
  source 1..19: mol_type = other RNA
  modified_base order(1..6,10..19): mod_base = OTHER; note = 2-Prime-O-Methyl RNA nucleotides
  modified_base 7..9: mod_base = OTHER; note = 2-Prime-deoxy-2-prime-F RNA nucleotides
  modified_base 17..19: mod_base = OTHER; note = Phosphorothioate
cgtacgcggaatacttcga

SEQ ID NO: 3 - RNA, 19 nt, synthetic construct
  source 1..19: mol_type = other RNA
  modified_base order(1,3,5,7,9,11,13,15,17,19): mod_base = OTHER; note = 2-Prime-O-Methyl RNA nucleotides
  modified_base order(2,4,6,8,10,12,14,16,18): mod_base = OTHER; note = 2-Prime-deoxy-2-prime-F RNA nucleotides
  modified_base order(1..3,17..19): mod_base = OTHER; note = Phosphorothioate
aaccagaagaagcaggtga

SEQ ID NO: 4 - RNA, 19 nt, synthetic construct
  source 1..19: mol_type = other RNA
  modified_base order(1..6,10..19): mod_base = OTHER; note = 2-Prime-O-Methyl RNA nucleotides
  modified_base 7..9: mod_base = OTHER; note = 2-Prime-deoxy-2-prime-F RNA nucleotides
  modified_base 17..19: mod_base = OTHER; note = Phosphorothioate
tcacctgcttcttctggtt

SEQ ID NO: 5 - RNA, 19 nt, synthetic construct
  source 1..19: mol_type = other RNA
  modified_base order(1,3,5,7,9,11,13,15,17,19): mod_base = OTHER; note = 2-Prime-deoxy-2-prime-F RNA nucleotides
  modified_base order(2,4,6,8,10,12,14,16,18): mod_base = OTHER; note = 2-Prime-O-Methyl RNA nucleotides
  modified_base 17..19: mod_base = OTHER; note = Phosphorothioate
tcacctgcttcttctggtt

SEQ ID NO: 6 - RNA, 19 nt, synthetic construct
  source 1..19: mol_type = other RNA
  modified_base order(1,3,5,7,9,11,13,15,17,19): mod_base = OTHER; note = 2-Prime-deoxy-2-prime-F RNA nucleotides
  modified_base order(2,4,6,8,10,12,14,16,18): mod_base = OTHER; note = 2-Prime-O-Methyl RNA nucleotides
  modified_base 17..19: mod_base = OTHER; note = Phosphorothioate
cgtacgcggaatacttcga

SEQ ID NO: 7 - RNA, 19 nt, synthetic construct
  source 1..19: mol_type = other RNA
tcgaagtattccgcgtacg

SEQ ID NO: 8 - RNA, 19 nt, synthetic construct
  source 1..19: mol_type = other RNA
cgtacgcggaatacttcga

SEQ ID NO: 9 - RNA, 19 nt, synthetic construct
  source 1..19: mol_type = other RNA
aaccagaagaagcaggtga

SEQ ID NO: 10 - RNA, 19 nt, synthetic construct
  source 1..19: mol_type = other RNA
tcacctgcttcttctggtt

(19) United States
(12) Patent Application Publication
(10) Pub. No.: US 2026/0275350 A1
(43) Pub. Date: Sep. 17, 2026

(54) COMPOUNDS AND COMPOSITIONS FOR USE IN STEM CELL TRANSPLANTATION

(71) Applicants: SILENCE THERAPEUTICS GMBH, Berlin (DE); New York Blood Center, Inc., New York, NY (US)

(72) Inventors: Ute Schaeper, Berlin (DE); Francesca Vinchi, New York, NY (US)

(21) Appl. No.: 19/168,653

(22) PCT Filed: Mar. 27, 2024
(86) PCT No.: PCT/EP2024/058252

§ 371 Date: Sep. 24, 2025

Related U.S. Application Data
(60) Provisional application No. 63/454,779, filed on Mar. 27, 2023.

(51) Int. Cl. C12N 15/113; A61P 35/00

(52) U.S. Cl. CPC C12N 15/1137; A61P 35/00; C12Y 304/21109; C12N 2310/14; C12N 2310/351

(57) ABSTRACT

The invention relates to a Matriptase-2 (MT2) inhibitor, TMPRSS6 inhibitor, Ferroportin blocker, or hepcidin enhancer. In particular, the invention relates to the use of compounds capable of reducing at least one of systemic iron level, Transferrin saturation (Tsat), non-transferrin-bound iron (NTBI), and labile plasma iron (LPI). This may take place by inhibiting the expression of a target gene, wherein the target gene may be Transmembrane protease, serine 6 (TMPRSS6). Further, the invention relates to compositions comprising said compound and to methods for using such compounds and/or compositions. The uses may comprise therapeutic uses such as for reduction or iron overload and the prevention of iron related toxicity before, during or after chemotherapeutic conditioning, stem cell transplantation and engraftment.

### FIELD OF THE INVENTION

The invention relates to the use of a Matriptase-2 (MT2) inhibitor, TMPRSS6 inhibitor, Ferroportin blocker, or hepcidin enhancer. In particular, the invention relates to compounds capable of at least one of: reducing systemic iron level, Transferrin saturation (Tsat), non-transferrin-bound iron (NTBI), and labile plasma iron (LPI). This may take place by inhibiting the expression of a target gene, wherein the target gene may be Transmembrane protease, serine 6 (TMPRSS6). Further, the invention relates to compositions comprising said compound and to methods for using such compounds and/or compositions. The uses may comprise therapeutic uses such as for reduction of iron overload and the prevention of iron related toxicity before, during or after cell explantation, implantation or transplantation related therapies, including chemotherapeutic conditioning, stem cell transplantation, and engraftment.

### BACKGROUND

Stem cell transplantation (SCT), i.e., the transplantation of stem cells to a subject in need thereof, is the only option for curative treatment for some patients with diseases including but not limited to hematopoietic stem cell transplantation (HSCT) for treatment of leukemias, such as AML, or other hematological diseases, such as beta-thalassemia, sickle cell disease, and bone marrow failure. Certain other conditions resulting from chemotherapeutic or irradiation therapies, such as myeloablation, also benefit from SCT. Similarly, high dose therapy and autologous HSCT is a treatment option for patients with selective hematologous and non-hematologous tumors. In addition, gene therapies to correct the defects of certain hematological genetic diseases commonly require bone marrow ablation prior to engraftment of gene modified autologous stem cells.

The myelodysplastic syndromes (MDS), formerly known as preleukemia because they might develop into leukemia, are a diverse collection of hematological medical conditions that involve ineffective production (or dysplasia) of the myeloid class of blood cells. The myelodysplastic syndromes are all disorders of the hematopoietic stem cells in the bone marrow. In MDS, hematopoiesis (blood production) is disorderly and ineffective. The number and quality of blood-forming cells decline irreversibly in MDS, further impairing blood production. Patients with MDS can develop severe anemia and require blood transfusions. In some cases, the disease worsens, and the patient develops cytopenias (low blood counts) caused by progressive bone marrow failure. Since MDS are stem cell related conditions, HSCT offers the potential for curative therapy, particularly in more severely affected patients.

Despite a better understanding of transplant immunology and improved graft-versus-host disease (GVHD) prophylaxis as well as in improvement of supportive care, there are still many risks associated with such transplantations and there is still significant morbidity and mortality associated with HSCT (Evens et al. Bone Marrow Transplantation 2004, 34, 561-571). Toxicities mainly result from organ damage induced by the preparative regimen, neutropenia predisposing to bacterial or fungal infections, or to impaired cellular immunity that makes patients vulnerable to viral and other opportunistic infections.

Iron overload, primarily due to transfusion requirement and ineffective erythropoiesis, is common but often remains uncontrolled during HSCT, potentially affecting the outcome of transplants as well as gene therapy protocols. Iron overload may contribute to treatment related toxicity and mortality after transplantation (Evens et al. Bone Marrow Transplantation 2004, 34, 561-571). Patients selected for HSCT frequently present with iron overload and elevated transferrin saturation due to a history of recurrent blood transfusions. Pre-existing iron overload is an adverse prognostic factor for patients undergoing HSCT (Wermke et al, 2012, Clin. Cancer Res, 18 (23), Atilla et al. Turk J Hematol 2017; 34:1-9). Indeed, elevated ferritin, transferrin saturation, hepatic iron as well as non-transferrin bound iron (NTBI) emerged as adverse prognostic factors for post-transplant survival and complications in β-thalassemia and also in sickle cell disease, MDS, and leukemia. Furthermore, it was recently shown that conditioning prior to HSC transplantation further exacerbates the presence of circulating ‘free’ non-transferrin-bound iron (NTBI) and labile plasma iron (LPI), the reactive fraction of LPI (Naoum et al. Hematol. Oncol. Stem Cell Ther 2016 9, 165-167). Importantly, NTBI positivity predicted an inferior overall survival in transplanted patients with myelodysplastic syndrome MDS or AML (Wermke et al Lancet Haematol. 2018 May; 5(5):e201-e210reference).

### Current Treatment Options to Prevent Iron Overload to Reduce Iron Mediated Toxicity Before or During Transplantation and Engraftment of HSC: Phlebotomy

Humans lack a dedicated iron excretion mechanism and iron loss occurs passively through sloughing of the skin and intestinal epithelium as well as the urine. Under normal conditions loss of 1 to 2 mg iron/day is balanced by absorption of similar amount of uptake via the diet (Isidori et al Transplant Cell Ther. 2021 May; 27(5):371-379.). Iron overload thus will develop due to repeated blood transfusions or by dysregulated iron homeostasis due to reduced hepcidin levels or reduced hepcidin activity, like in iron loading anemias or hereditary hemochromatosis (Camaschella et al. 2020 Vol. 105 No. 2 (2020): February 2020). Phlebotomy is considered the simplest approach to remove excess iron from the body. In HSCT recipients, phlebotomy could be an option to remove excess iron after successful engraftment of the hematopoietic stem cells, but not during conditioning and engraftment phase and while patients still require blood transfusions. In general, this approach is usually not applied due to the commonly present anemic condition in patients undergoing HSCT.

### Chelation

Iron chelation therapy is currently the pharmacologic option for alleviating iron burden in patients with iron overload (reviewed by Isidori et al 2021 Transplant Cell Ther. 2021 May; 27(5):371-379): Deferoxamine is not indicated in this setting because of its short half-life which requires prolonged infusion, and its siderophore activity, which allows for iron release to micro-organisms. The oral chelator deferiprone has a longer half-life but is also associated with the potential to develop agranulocytosis. The latest generation chelator, deferasirox has the advantage of a longer half-life and the ability to effectively scavange NTBI/LPI. Some common adverse events associated with deferasirox therapy may overlap with acute post-HSCT adverse effects and therefore may restrict its use to better manage late post-transplant iron toxicity to improve long-term patient outcome.
Ref: Essmann S, Heestermans M, Dadkhah A, Janson D, Wolschke C, Ayuk F, Kröger NM, Langebrake C. Iron Chelation with Deferasirox Suppresses the Appearance of Labile Plasma Iron During Conditioning Chemotherapy Prior to Allogeneic Stem Cell Transplantation. Transplant Cell Ther. 2023 January; 29(1):42.e1-42.e6. doi: 10.1016/j.jtct.2022.10.002. Epub 2022 Oct. 12. PMID: 36241148.

### SUMMARY OF THE INVENTION

In one aspect the invention relates to a therapeutic agent for use in the treatment of an iron metabolism disease or condition wherein the therapeutic agent comprises an inhibitor selected from:
(a) a TMPRSS6 inhibitor;
(b) a MT2 inhibitor; or
(c) a Ferroportin inhibitor,
   and wherein the treatment comprises cell explantation, implantation, or transplantation.

In certain aspects, the treatment of the iron metabolism disease or condition further comprises the step of cell explantation, implantation, or transplantation therapy. In certain aspects, it comprises stem cell transplantation, preferably HSCT.

In certain aspects, the therapeutic agent or pharmaceutical composition of the invention is administered to the subject in need thereof prior to, during or after conditioning for HSCT. In certain aspects, the therapeutic agent or pharmaceutical composition of the invention is administered to the subject in need thereof prior to conditioning for HSCT.

In certain aspects, the therapeutic agent or pharmaceutical composition of the invention is administered to the subject in need thereof prior to, during or after receiving HSCT. In certain aspects, the therapeutic agent or pharmaceutical composition of the invention is administered to the subject in need thereof prior to receiving HSCT.

In another aspect the invention relates to a pharmaceutical composition for use in the treatment of an iron metabolism disease or condition, the pharmaceutical composition comprising an effective amount of the therapeutic agent as defined in any of the preceding claims, further comprising a pharmaceutically acceptable diluent, carrier, or excipient.

In another aspect the invention relates to a method of treating an iron metabolism disease or condition in a subject in need thereof, comprising administering an effective amount of the pharmaceutical composition or the therapeutic agent of the invention, wherein the treatment comprises cell explantation, implantation, or transplantation.

In another aspect the invention relates to the use of a therapeutic agent or a pharmaceutical composition of the invention for the manufacture of a medicament for the treatment of an iron metabolism disease or condition, wherein the treatment comprises cell explantation, implantation, or transplantation.

### DETAILED DESCRIPTION OF THE INVENTION

### New Treatment Option: Inhibiting Matriptase-2/TMPRSS6

The inventors have surprisingly found a different treatment option to reduce transferrin saturation, which further prevents NTBI, iron overload, and reduce iron mediated toxicity before or during transplantation and engraftment of HSC. They have found that an inhibitor of the TMPRSS6 gene, an inhibitor of the protein Matriptase-2 (MT2), a ferroportin blocker or a hepcidin enhancer, can be used for such treatment. MT2 is the protein product of the TMPRSS6 gene and is a type II transmembrane serine protease that plays a critical role in the regulation of iron homeostasis. MT2 is a negative regulator for synthesis induction of the peptide hormone hepcidin. Hepcidin is a peptide hormone predominantly produced by the liver and acts as a negative regulator of gastro-intestinal iron absorption and iron release from storage, preventing iron efflux from enterocytes and macrophages into the circulation. Hepcidin regulates iron balance in the body by blocking the cellular release of iron via the only known cellular iron export protein, ferroportin. Elevated hepcidin levels can thereby induce iron restriction, reducing iron availability within the body (McDonald et al, American Journal of Physiology, 2015, vol 08 no. 7, C539-C547). TMPRSS6 is primarily expressed in the liver, although high levels of TMPRSS6 mRNA are also found in the kidney, with lower levels in the uterus and much lower amounts detected in many other tissues (Ramsay et al, Haematologica (2009), 94(*6), 84-849).

Inhibition of MT2 by reducing the expression of the TMPRSS6 gene appears to be a particularly promising approach. Inhibition of TMPRSS6 expression can be attained in several ways. One way is the use of an inhibitory nucleic acid such as a siRNA or an antisense oligonucleotide (ASOs). These are short nucleic acids that inhibit the formation of proteins by causing targeted degradation of the mRNA molecules that encode these proteins. Such gene silencing agents are becoming increasingly important for therapeutic applications in medicine. For the pharmaceutical development of such nucleic acids, it is among others necessary that they can be synthesised economically, are metabolically stable, are specifically targeted to a tissue, are able to enter cells and function within acceptable limits of toxicity.

Double-stranded RNAs (dsRNA) able to bind through complementary base pairing to expressed mRNAs have been shown to block gene expression (Fire et al., 1998, Nature. 1998 Feb. 19; 391(6669):806-11 and Elbashir et al., 2001, Nature. 2001 May 24; 411(6836):494-8) by an endogenous mechanism that has been termed “RNA interference (RNAi)”. Short dsRNAs direct gene specific, post-transcriptional silencing in many organisms, including vertebrates, and have become a useful tool for studying gene function. RNAi is mediated by the RNA induced silencing complex (RISC), a sequence specific, multi component nuclease that degrades targeted messenger RNAs having sufficient complementary or homology to the silencing trigger strand loaded as an siRNA duplex into the RISC complex.

Inhibition of TMPRSS6 expression in wild type mice as well as animal models for hereditary hemochromatosis and beta-thalassemia by GalNAc TMPRSS6 siRNA molecules raised serum hepcidin levels and reduced serum iron and transferrin saturation (Altamura et al. Hemasphere. 2019 December; 3(6): e301., Vadolas et al. Br. J. Hematology 2021 July; 194(1):200-210). Duration of action of highly active TMPRSS6 siRNA sequences is enhanced by specific chemical modification of the GalNAc siRNA compound leading to infrequent dosing requirements (once every 3-5 weeks) to induce iron restriction by hepcidin induction (Altamura et al. Hemasphere. 2019 December; 3(6): e301., Vadolas et al. Br. J. Hematology 2021 July; 194(1):200-210).

Benefits of TMPRSS6 siRNA Mediated Therapy

Targeting MT2 or TMPRSS6 has advantages compared to all the treatment options listed above.

TMPRSS6 siRNA Versus Phlebotomy:

Patients undergoing chemotherapeutic condition and preparation for HSC-T may become anemic and may depend on blood transfusions. Phlebotomy lowers haematocrit and haemoglobin levels by removing red blood cells from the circulation, which will worsen the anemia. This could be avoided by the new treatment method, as iron restriction through elevation of endogenous hepcidin levels via a MT2 inhibitor, such as a TMPRSS6 siRNA, leads to a redistribution of iron within the body. This allows to limit the release of serum iron, NTBI or LPI during or after conditioning.

Phlebotomy is also associated with side effects linked to fluid shifts, including dizziness, nausea, and vasovagal syncope. Therapies based on MT2 inhibition, such as TMPRSS6 siRNA therapy, are unlikely to affect fluid levels compartments and these complications would therefore likely not arise with this therapeutic approach.

TMPRSS6 siRNA Versus Chelators:

Induction of endogenous hepcidin by inhibiting TMPRSS6 expression through GalNAc siRNAs thereby presents a promising therapeutic strategy to reduce toxicity during and after conditioning, enhance engraftment, and reduce non-relapse morbidity and mortality in patients receiving HSCT.

Current treatments of disorders with prognosed elevated ferritin, transferrin saturation, hepatic iron as well as non-transferrin bound iron (NTBI) such as beta-thalassemia, sickle cell disease, MDS, and leukemia all have drawbacks and are often so toxic to the host, that they are contraindicated for large groups of patients and/or cannot be provided in sufficient amounts to prevent GVHD. There is therefore a clear need for decreasing the risk associated with HSCT and increasing its effectiveness for various disorders. The invention addresses this need. The use of an MT2 inhibitor, such as a GalNAc-conjugated TMPRSS6 siRNA, presents a promising therapeutic strategy for disorders with elevated ferritin, transferrin saturation, hepatic iron as well as non-transferrin bound iron.

The inventors have surprisingly found that reduction of iron overload or reduction of labile plasma iron before, during, and after transplantation and engraftment benefits patients undergoing HSC transplantation.

One aspect of the present invention relates to the use of a therapeutic agent comprising or consisting of:
(a) a TMPRSS6 inhibitor;
(b) a MT2 inhibitor; or
(c) a ferroportin inhibitor.

In one aspect, the present invention relates to a therapeutic agent for use in treating a subject prior to, during, or after receiving a cell explantation, implantation, or transplantation, wherein the therapeutic agent comprises an inhibitor selected from the group consisting of:
(a) a TMPRSS6 inhibitor;
(b) a MT2 inhibitor; or
(c) a ferroportin inhibitor,
   wherein the cell explantation, implantation, or transplantation may be a stem cell transplantation and optionally wherein the treatment further comprises co-administration of one or more chemotherapeutic agent, radiotherapy, and/or immunotherapy.

In one aspect, the present invention relates to a therapeutic agent for use as a medicament for reducing systemic iron levels, transferrin saturation, non-transferrin-bound iron (NTBI), and/or labile plasma iron (LPI) in a subject,

wherein said subject exhibits at least one of systemic iron overload, transferrin saturation, non-transferrin-bound iron (NTBI) overload, and labile plasma iron (LPI) overload, wherein the therapeutic agent comprises an inhibitor selected from:
(a) a TMPRSS6 inhibitor;
(b) a MT2 inhibitor; or
(c) a Ferroportin inhibitor,
   wherein the treatment further comprises a cell explantation, implantation, or transplantation.

In one aspect, the present invention relates to a therapeutic agent for use in treating a subject prior to, during, or after a cell explantation, implantation, or transplantation, wherein the therapeutic agent comprises an inhibitor selected from the group consisting of:
(a) a TMPRSS6 inhibitor;
(b) a MT2 inhibitor; or
(c) a ferroportin inhibitor.

In one aspect, the present invention relates to a therapeutic agent for use in treating or preventing systemic iron overload, transferrin saturation, non-transferrin-bound iron (NTBI) overload, and/or labile plasma iron (LPI) overload in a subject in need thereof, wherein the therapeutic agent comprises an inhibitor selected from:
(a) a TMPRSS6 inhibitor;
(b) a MT2 inhibitor; or
(c) a Ferroportin inhibitor,
   wherein the treatment further comprises the step of cell explantation, implantation, or transplantation.

In one aspect, the present invention relates to a therapeutic agent for use in the treatment of an iron metabolism disease or condition, wherein the therapeutic agent comprises an inhibitor selected from:
a TMPRSS6 inhibitor;
a MT2 inhibitor; or
a Ferroportin inhibitor
wherein the treatment further comprises the step of cell explantation, implantation, or transplantation and optionally wherein the treatment further comprises co-administration of one or more chemotherapeutic agent, radiotherapy, and/or immunotherapy.

In one aspect, the present invention relates to a therapeutic agent for use in the treatment of an iron metabolism disease or condition associated with at least one of systemic iron overload, transferrin saturation, non-transferrin-bound iron (NTBI) overload, and labile plasma iron (LPI) overload, wherein the therapeutic agent comprises an inhibitor selected from:
a TMPRSS6 inhibitor;
a MT2 inhibitor; or
a Ferroportin inhibitor,
wherein the treatment comprises the step of the step of cell explantation, implantation, or transplantation.

In one aspect, the present invention relates to a therapeutic agent for use in treating a disease or condition that would benefit from the reduction of at least one of: systemic iron level, transferrin saturation (Tsat), non-transferrin-bound iron (NTBI), and labile plasma iron (LPI), wherein the therapeutic agent comprises an inhibitor selected from the group consisting of:
(a) a TMPRSS6 inhibitor;
(b) a MT2 inhibitor; or
(c) a ferroportin inhibitor.

In certain aspects, the iron metabolism disease or condition is one that will benefit from the reduction of iron as measured by one or more of systemic iron level, transferrin saturation (Tsat), non-transferrin-bound iron (NTBI), or labile plasma iron (LPI).

In certain aspects, the iron metabolism disease or condition exhibits at least one of systemic iron overload, transferrin saturation, non-transferrin-bound iron (NTBI) overload, and labile plasma iron (LPI) overload.

In one aspect of the present invention, the agent that reduces measurable iron is selected from the group consisting of:
(i) antibody or antigen-binding fragments thereof, or a variant, fusion or derivative of said antibody or antigen-binding fragments, or a fusion of said variants, or derivatives thereof;
(ii) antibody mimics or mimetics (for example, based on non-antibody scaffolds);
(iii) RNA aptamer;
(iv) small molecule;
(v) CovX-body;
or
(vi) nucleic acid.

In one aspect of the invention, the antibody or antigen-binding fragment thereof (or a variant, fusion or derivative of said antibody or antigen-binding fragment), or a fusion of said variant or derivative thereof of (i); and/or the antibody mimics of (ii) is selected from the group consisting of affibodies, tetranectins, adnectins, monobodies, anticalins, DARPins, ankyrins, avimers, iMabs, microbodies peptide aptamers, Kunitz domains, aflilins, and any combination thereof.

Several TMPRSS6, MT2, and ferroportin inhibitors which may be used for the purpose of the present invention are known in the art. The following are some examples of publications which disclose some of the mentioned inhibitors:
a) for MT2: Gütschow et al. J. Med. Chem. 2010, 53, 15, 5523-5535; Marsault et al., European Journal of Medicinal Chemistry, Volume 129, 2017, Pages 110-123, Stirnberg et. Al. Pharmaceuticals 2018, 11(2), 49, all of which are incorporated by reference herein for all they disclose regarding MT2 inhibitors;
b) for TMPRSS6: WO2018/185240A1, WO2014190157A1, WO2016085852A1; WO2022/231999A1, WO2016161429A1, all of which are incorporated by reference herein for all they disclose regarding TMPRSS6 inhibitors;
c) for ferroportin: WO2022157185A1, WO2017/068089, WO2017/068090, WO2018/192973, WO2017/068089 and WO2017/068090, all of which are incorporated by reference herein for all they disclose regarding ferroportin inhibitors. Further, such as hepcidin mimetics, which work as ferroportin blockers are known.

The skilled person knows TMPRSS6, MT2, and/or ferroportin inhibitors. Any of the TMPRSS6, MT2, and/or ferroportin inhibitors known in the art may be used by the skilled person for the purpose of the present invention, which is their use for treating an iron metabolism disease or condition that exhibits at least one of systemic iron overload, transferrin saturation, non-transferrin-bound iron (NTBI) overload, and/or labile plasma iron (LPI) overload.

In one aspect of the invention, the therapeutic agent for use is a nucleic acid, wherein the nucleic acid comprises at least one duplex region that comprises at least a portion of a first strand and at least a portion of a second strand that is at least partially complementary to the portion of the first strand, wherein said first strand is at least partially complementary to at least a portion of RNA transcribed from the TMPRSS6 gene and is capable of inhibiting expression of TMPRSS6.

In certain aspects, one or more nucleotides on the first and/or second strand are modified, to form modified nucleotides.

In certain aspects, the nucleic acid is conjugated to a ligand, optionally at the 5′ end of the second strand.

In certain aspects, the ligand comprises (i) one or more N-acetyl galactosamine (GalNAc) moieties and derivatives thereof, and (ii) a linker, wherein the linker conjugates the GalNAc moieties to the nucleic acid.

In certain aspects, the linker is a bivalent or trivalent or tetravalent branched structure.

In certain aspects, the nucleic acid is stabilised at the 5′ and/or 3′ end of either or both strands. In certain aspects the nucleic acid comprises a phosphorothioate linkage between the terminal one, two or three 3′ nucleotides and/or 5′ nucleotides of the first and/or the second strand or comprising a phosphorodithioate linkage.

In certain aspects, the nucleic acid comprises two phosphorothioate linkage between each of the three terminal 3′ and between each of the three terminal 5′ nucleotides on the first strand, and two phosphorothioate linkages between the three terminal nucleotides of the 3′ end of the second strand.

In certain aspects, the unmodified or modified nucleic acid may be selected from Table 1a:

| | SEQ ID No. | SEQ ID No. |
|---|---|---|
| | unmodified | corresponding |
| Duplex ID | sequence | modified sequence |
| TMPRSS6-hcr-26 | 436 | 213 |
| | 437 | 214 |
| TMPRSS6-hcm-9 | 333 | 17 |
| | 334 | 18 |
| TMPRSS6-h-7 | 329 | 13 |
| | 330 | 14 |
| TMPRSS6-h-2 | 319 | 3 |
| | 320 | 4 |
| TMPRSS6-h-5 | 325 | 9 |
| | 326 | 10 |
| TMPRSS6-SR21 | 390 | 167 |
| | 391 | 168 |
| TMPRSS6-hc-16 | 418 | 195 |
| | 419 | 196 |
| TMPRSS6-hc-18 | 422 | 199 |
| | 423 | 200 |
| TMPRSS6-hc-1 | 317 | 1 |
| | 318 | 2 |
| TMPRSS6-hcmr-15 | 345 | 29 |
| | 346 | 30 |

as disclosed in WO2018/185240A1. The unmodified or modified nucleic acids may be combined with a ligand as known in the art and for example as disclosed in the same document as GN3-TMPRSS6-hcm9 or GN3-TMPRSS6-hc18. The skilled person will appreciate that any of the unmodified or modified sequences may be combined with any linker known in the art, such as for example a GalNAc linker.

In certain aspects, the nucleic acid may be selected from Table 1 b:

| SEQ ID | |
|---|---|
| NO | Name |
| 285 | STS12009V34L4-A |
| 286 | STS12009V34L4-B |
| 287 | STS12009V36L4-A |
| 288 | STS12009V36L4-B |
| 289 | STS12009V37L4-A |
| 290 | STS12009V37L4-B |
| 291 | STS12009V40L4-A |
| 292 | STS12009V40L4-B |
| 293 | STS12209V4L4-A |
| 294 | STS12209V4L4-B |
| 295 | STS12209V5L4-A |
| 296 | STS12209V5L4-B |
| 297 | STS12209L4-A |
| 298 | STS12209L4-B |
| 299 | STS12209V1L4-A |
| 300 | STS12209V1L4-B |

as disclosed in WO2018/185240A1. The nucleic acids may be combined with any ligand known in the art. The skilled person will appreciate that any of the sequences named above may be combined with any linker known in the art, such as for example a GalNAc linker and as for example disclosed in the same document under the SEQ ID Nos. named above, i.e., for example SED ID NO 286, 288, 290, 292, etc.

In certain aspects, the unmodified or modified nucleic acid may be selected from Table 2:

| | SEQ ID No. | SEQ ID No. |
|---|---|---|
| | unmodified | corresponding |
| Duplex ID | sequence | modified sequence |
| EU400 | see unmodified counterpart | 1 |
| | under 1 | |
| | see unmodified counterpart | 2 |
| | under 2 | |
| EU401 | 6 | 3 |
| | 7 | 1386 |
| EU402 | 6 | 3 |
| | see unmodified counterpart | 1387 |
| | under 5 | |

as disclosed in WO2022229150A1. The nucleic acids may be unmodified or modified and said unmodified or modified nucleic acids may be combined with a ligand as known in the art and for example as disclosed in the same document. The skilled person will appreciate that any of the unmodified or modified sequences may be combined with any linker known in the art, such as for example a GalNAc linker. The same document discloses the modified sequences of EU400, EU401, and EU402 including a linker under SEQ ID NO: 2, 4, and 5 correspondingly.

In certain aspects, the unmodified or modified nucleic acid may be selected from Table 3:

| | | SEQ ID No. |
|---|---|---|
| | SEQ ID No. | corresponding |
| | unmodified | modified |
| Duplex ID | sequence | sequence |
| AD-1556360 | 119 | 371 |
| | 245 | 497 |
| AD-1570930.1 | 654 | 1873 |
| | 1726 | 2100 |
| AD-1570931.1 | 1662 | 1874 |
| | 1727 | 2101 |
| AD-1570932.1 | 28 | 1875 |
| | 1728 | 2102 |
| AD-1570951.1 | 1667 | 1894 |
| | 1739 | 2121 |
| AD-1570972.1 | 1673 | 1915 |
| | 1755 | 2142 |
| AD-1571033.1 | 1686 | 1974 |
| | 1790 | 2203 |
| AD-1571047.1 | 668 | 1988 |
| | 1801 | 2217 |
| AD-1571052.1 | 676 | 1992 |
| | 1803 | 2221 |
| AD-1571061.1 | 1697 | 2001 |
| | 1809 | 2230 |
| AD-1571074.1 | 721 | 2013 |
| | 916 | 2242 |
| AD-1571075.1 | 1704 | 2014 |
| | 1818 | 2243 |
| AD-1571076.1 | 1705 | 2015 |
| | 919 | 2244 |
| AD-1571078.1 | 59 | 2017 |
| | 185 | 2246 |
| AD-1571105.1 | 773 | 2042 |
| | 1836 | 2271 |
| AD-1571155.1 | 838 | 2092 |
| | 1865 | 1653 |
| AD-1571157.1 | 1725 | 2094 |
| | 1867 | 2323 |
| AD-1571158.1 | 844 | 2095 |
| | 1868 | 2324 |
| AD-1571159.1 | 845 | 2096 |
| | 1869 | 2325 |
| AD-1571160.1 | 846 | 2097 |
| | 1870 | 2326 |

as disclosed in WO2022231999A1. The nucleic acids may e unmodified or modified and said unmodified or modified nucleic acids may be combined with a ligand as known in the art and for example as disclosed in the same document. The skilled person will appreciate that any of the unmodified or modified sequences may be combined with any linker known in the art, such as for example a GalNAc linker.

In certain aspects, the unmodified or modified nucleic acid may be selected from Table 4:

SEQ ID No.
615903
630718
630722
647393
647395
647419
647420
647446
647447
647448
647449
647475
647476
647477
647478
647506
702843
705051
706940
706942

as disclosed in WO2016161429A1. The nucleic acids may be unmodified or modified and said unmodified or modified nucleic acids may be combined with a ligand as known in the art and for example as disclosed in the same document. The skilled person will appreciate that any of the unmodified or modified sequences may be combined with any linker known in the art, such as for example a GalNAc linker.

Synthesis of (vp)-mU-phos can be performed as described in Prakash, Nucleic Acids Res. 2015, 43(6), 2993-3011 and Haraszti, Nucleic Acids Res. 2017, 45(13), 7581-7592. Synthesis of the phosphoramidite derivatives of ST23 (ST23-phos) and similar can be performed as described in WO2017/174657.

In certain aspects, the conjugated nucleic acid is conjugated to a triantennary ligand with one of the following structures:

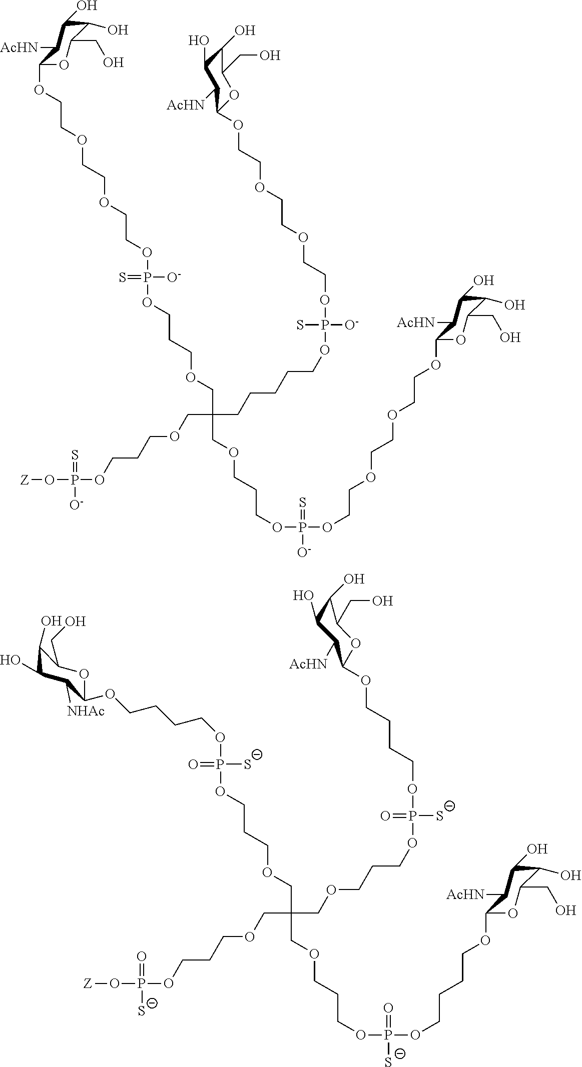

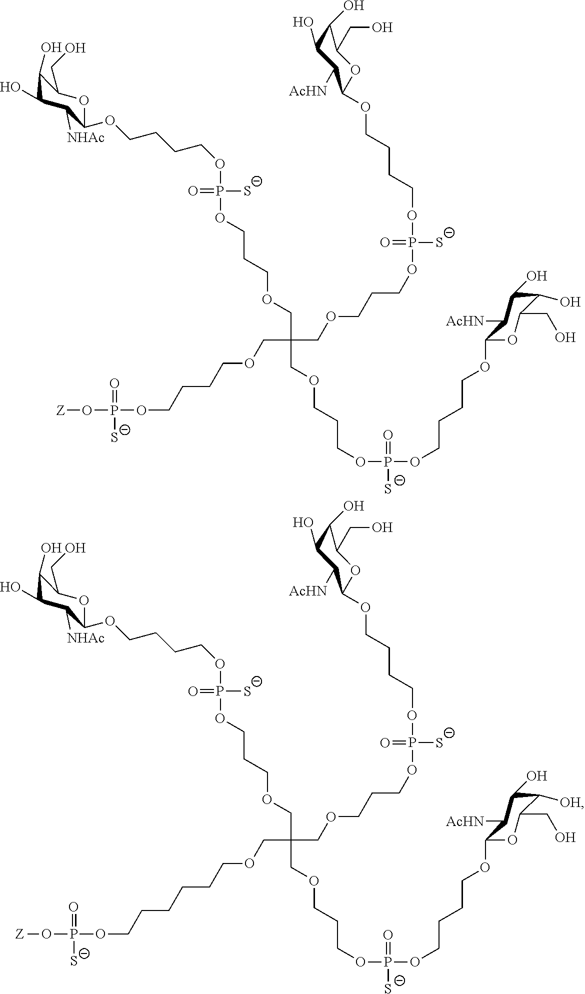

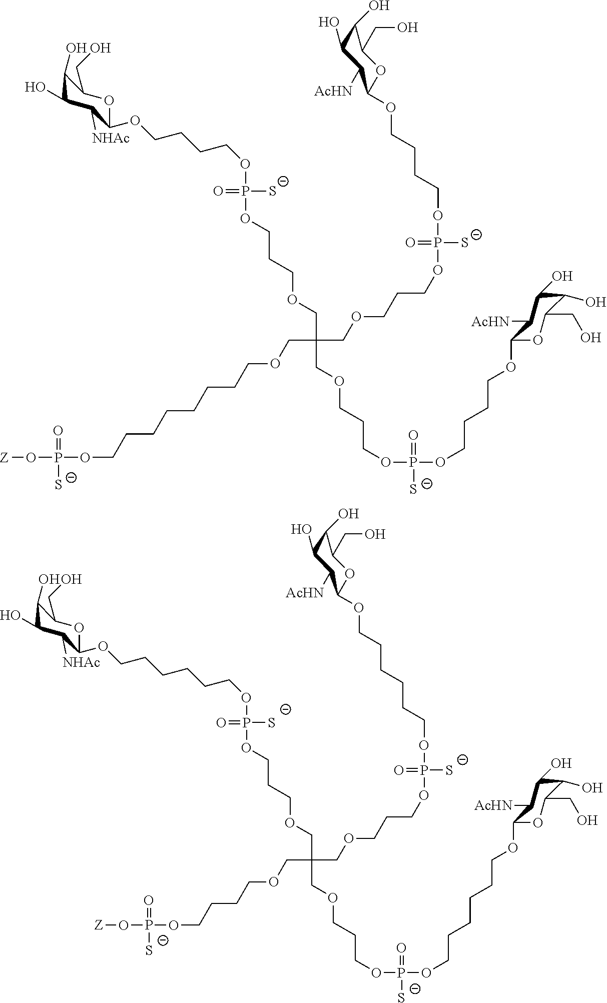

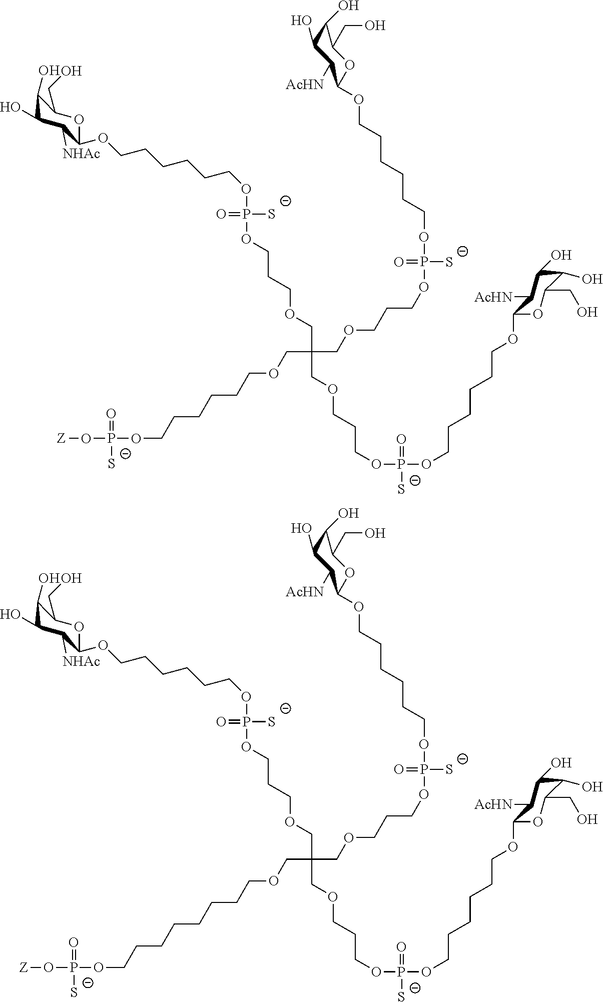

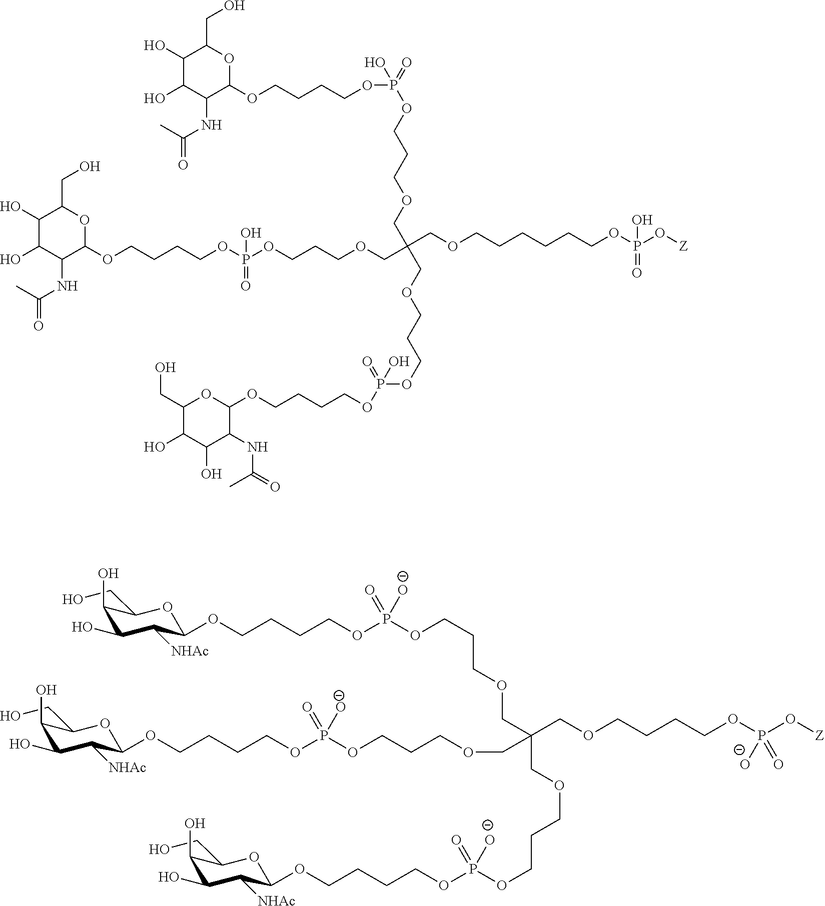

wherein Z is any nucleic acid as defined herein.

In one aspect, the conjugated nucleic acid, is conjugated to a triantennary ligand with the following structure:

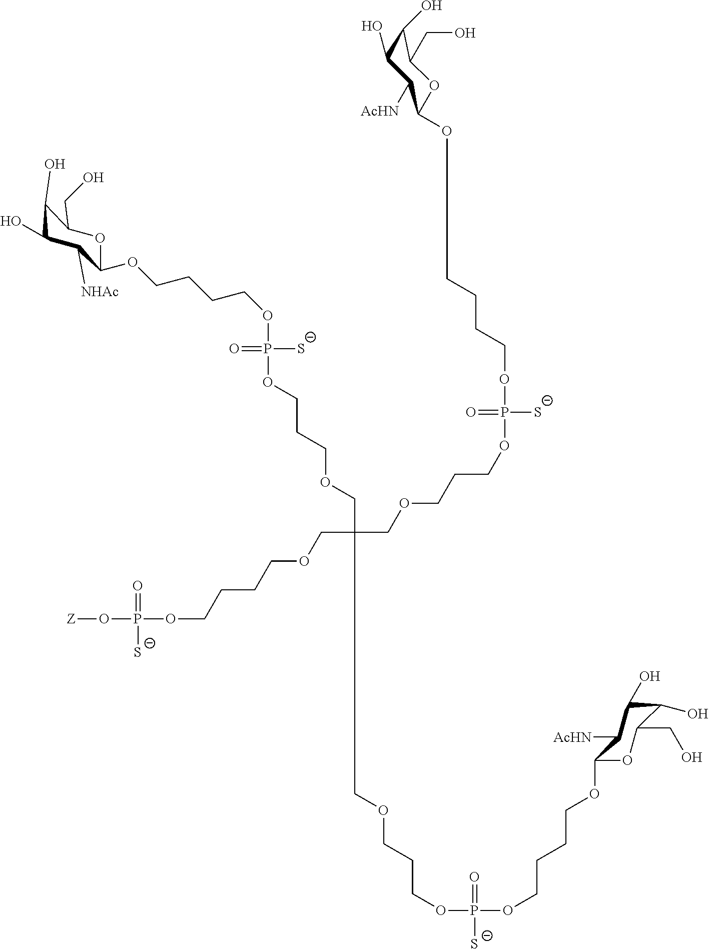

wherein Z is any nucleic acid as defined herein and wherein the terminal phosphorothioate group of the ligand moiety is bonded to the 5′position of the 5′terminal nucleotide of the second strand of the nucleic acid (which is denoted by the “Z”) or wherein the terminal phosphorothioate group of the ligand moiety is bonded to the 3′position of the 3′terminal nucleotide of the second strand of the nucleic acid (“Z”).

In certain aspects, the nucleic acid (which is denoted by the “Z”) is conjugated to the (triantennary) ligand via the phosphate or thiophosphate group of the ligand moiety which links the ligand to the 5′position of the 5′terminal nucleotide of the second strand of the nucleic acid or which links the ligand to the 3′position of the 3′terminal nucleotide of the second strand of the nucleic acid.

A ligand of formula (II), (III) or (IV) or any one of the triantennary ligands disclosed herein can be attached at a nucleic acid end or to a nucleotide that is not at the end of the nucleic acid. In the case of a double-stranded nucleic acid, the ligand can be attached at the 3′-end of the first (antisense) strand and/or at any of the 3′ and/or 5′ end of the second (sense) strand. The nucleic acid can comprise more than one ligand of formula (II), (III) or (IV) or any one of the triantennary ligands disclosed herein. However, a single ligand of formula (II), (III) or (IV) or any one of the triantennary ligands disclosed herein is preferred because a single such ligand is sufficient for efficient targeting of the nucleic acid to the target cells. Preferably in that case, at least the last two, preferably at least the last three and more preferably at least the last four nucleotides at the end of the nucleic acid to which the ligand is attached are linked by a phosphodiester linkage.

In certain aspects, in the case of a double-stranded nucleic acid, the 5′-end of the first (antisense) strand is not attached to a ligand of formula (II), (III) or (IV) or any one of the triantennary ligands disclosed herein, since a ligand in this position can potentially interfere with the biological activity of the nucleic acid.

A nucleic acid with a single ligand of formula (II), (III) or (IV) or any one of the triantennary ligands disclosed herein at the 5′ end of a strand is easier and therefore cheaper to synthesise than the same nucleic acid with the same ligand at the 3′ end. Preferably therefore, a single ligand of any of formulae (II), (III) or (IV) or any one of the triantennary ligands disclosed herein is covalently attached to (conjugated with) the 5′ end a nucleic acid strand, and preferably to the 5′ end of the second strand when the nucleic acid is double-stranded.

In certain aspects, the nucleic acids of table 1a or 1b are used for the purpose of the present invention. In certain aspects, the nucleic acids of table 1b are used for the purpose of the present invention.

In certain aspects, the nucleic acids of table 2 are used for the purpose of the present invention. Additionally, a control molecule may be used, comprising a first strand comprising the modified sequence of SEQ ID NO: 1 of WO2022229150A1 and a second strand as follows: [ST23(ps)]3 C4XLT(ps) fC mG fU mA fC mG fC mG fG mA fA mU fA mC fU mU fC (ps) mG (ps) fA. Said molecule is named EU403 hereafter.

In certain aspects, the nucleic acid is EU401 or EU402. In certain aspects, the nucleic acid is EU401. In certain aspects, the nucleic acid is EU402. In certain aspects, EU401 and EU402 are interchangeable and can be used in combination.

In particular, EU400, EU401, EU402 and EU403 are as described in Table 5:

TABLE 5

| | | Sequence (A, | |
|---|---|---|---|
| | | first strand; | |
| | single | B, second | |
| Duplex | strand | strand, | modified sequence (A, first strand; B second |
| ID | ID | both 5′-3′) | strand, both 5′-3′) |
| EU400 | EU400A | UCGAAGUAUU | mU (ps)fC (ps) mG fA mA fG mU fA mU fU mC fC |
| | | CCGCGUACG | mG fC mG fU mA (ps)fC (ps) mG |
| | EU400B | CGUACGCGGA | [ST23(ps)]3 C6XLT(ps) mC mG mU mA mC mG fC |
| | | AUACUUCGA | fG fG mA mA mU mA mC mU mU mC (ps) mG (ps) |
| | | | mA |
| EU401 | EU401A | AACCAGAAGA | mA (ps) fA (ps) mC fC mA fG mA fA mG fA mA fG |
| | | AGCAGGUGA | mC fA mG fG mU (ps) fG (ps) mA |
| | EU401B | UCACCUGCUU | [ST23(ps)]3 C6XLT(ps) mU mC mA mC mC mU fG |
| | | CUUCUGGUU | fC fU mU mC mU mU mC mU mG mG (ps) mU (ps) |
| | | | mU |
| EU402 | EU401A | AACCAGAAGA | mA (ps) fA (ps) mC fC mA fG mA fA mG fA mA fG |
| | | AGCAGGUGA | mC fA mG fG mU (ps) fG (ps) mA |
| | EU402B | UCACCUGCUU | [STS23(ps)]3 C4XLT(ps)fU mC fA mC fC mU fG |
| | | CUUCUGGUU | mC fU mU fC mU fU mC fU mG fG (ps) mU (ps) fU |
| EU403 | EU400A | UCGAAGUAUU | mU (ps)fC (ps) mG fA mA fG mU fA mU fU mC fC |
| | | CCGCGUACG | mG fC mG fU mA (ps)fC (ps) mG |
| | EU403B | CGUACGCGGA | [ST23(ps)]3 C4XLT(ps) fC mG fU mA fC mG fC |
| | | AUACUUCGA | mG fG mA fA mU fA mC fU mU fC (ps) mG (ps) fA |

A skilled person knows that any of the known inhibiting nucleic acids may be modified or unmodified, and may be singly or multiply combined and conjugated with any of the known ligands, for examples as the ones mentioned above for the purpose of inhibiting the target gene.

In certain aspects, the nucleic acid is unmodified, which means is does not comprise, e.g., chemical modifications or conjugations known in the art. In certain aspects, the nucleic acid is chemically modified to enhance stability or other beneficial characteristics.

The nucleic acids can be synthesized or modified by methods well established in the art. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases, or conjugated bases; sugar modifications (e.g., at the 2′-position or 4′-position) or replacement of the sugar; or backbone modifications, including modification or replacement of the phosphodiester linkages. Terminal modifications can be added for a number of reasons, including to modulate activity or to modulate resistance to degradation. Terminal modifications useful for modulating activity include modification of the 5′ end with phosphate or phosphate analogs. Nucleic acids of the invention, on the first or second strand, may be 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (OH)₂(O)P-5′-CH₂—), 5′vinylphosphonate, 5′-alkyletherphosphonates (R=alkylether=methoxymethyl (MeOCH₂—), ethoxymethyl, etc., e.g., RP(OH)(O)—O-5′-). Examples of modifications are given in the documents mentioned above and further for example in WO2018185241.

Another modification of the nucleic acid involves linking the nucleic acid to one or more ligands, moieties or conjugates that enhance activity, cellular distribution, or cellular uptake of the nucleic acid e.g., into a cell. Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937). Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Ligands can also include targeting groups, e.g., a cell or tissue targeting agent, e.g., a lectin, glycoprotein, lipid, or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

In certain aspects, the conjugate is a carbohydrate conjugate. In certain embodiments, the carbohydrate is a monosaccharide. In certain embodiments, the monosaccharide is an N-acetylgalactosamine (GalNAc). GalNAc conjugates are described for example in WO2017/174657, US2021/0017214 and WO2022/231999.

In certain aspects, the invention relates to any nucleic acid, conjugated nucleic acid, nucleic acid for use, method, composition or use according to any disclosure herein, wherein the nucleic acid comprises a vinyl-(E)-phosphonate modification, such as a 5′ vinyl-(E)-phosphonate modification. In certain aspects, a 5′ vinyl-(E)-phosphonate modification in combination with a 2′-F modification at second position of the first strand. Vinyl-(E)-phosphonate 2′OMe-Uracil phosphoamidite can be synthesized and used in oligonucleotide synthesis according to literature published methods (Haraszti et al., Nuc. Acids Res., 45(13), 2017, 7581-7592).

In certain aspects, the nucleic acid of the present invention may include one or more phosphorothioate modifications on one or more of the terminal ends of the first and/or the second strand. Optionally, each or either end of the first strand may comprise one or two or three phosphorothioate modified nucleotides. Optionally, each or either end of the second strand may comprise one or two or three phosphorothioate modified nucleotides. Optionally, both ends of the first strand and the 5′ end of the second strand may comprise two phosphorothioate modified nucleotides. By phosphorothioate modified nucleotide it is meant that the linkage between the nucleotide and the adjacent nucleotide comprises a phosphorothioate group instead of a standard phosphate group.

In certain aspects, the invention relates to any nucleic acid, conjugated nucleic acid, nucleic acid for use, method, composition or use according to any disclosure herein, wherein the terminal nucleotide at the 3′ end of at least one of the first strand and the second strand is an inverted nucleotide and is attached to the adjacent nucleotide via the 3′ carbon of the terminal nucleotide and the 3′ carbon of the adjacent nucleotide and/or the terminal nucleotide at the 5′ end of at least one of the first strand and the second strand is an inverted nucleotide and is attached to the adjacent nucleotide via the 5′ carbon of the terminal nucleotide and the 5′ carbon of the adjacent nucleotide, optionally wherein
a. the 3′ and/or 5′ inverted nucleotide of the first and/or second strand is attached to the adjacent nucleotide via a phosphate group by way of a phosphodiester linkage; or
b. the 3′ and/or 5′ inverted nucleotide of the first and/or second strand is attached to the adjacent nucleotide via a phosphorothioate group or
c. the 3′ and/or 5′ inverted nucleotide of the first and/or second strand is attached to the adjacent nucleotide via a phosphorodithioate group.

Terminal modifications can also be useful for monitoring distribution, and in such cases the groups to be added may include fluorophores, e.g., fluorescein or an Alexa dye. Terminal modifications can also be useful for enhancing uptake, useful modifications for this include cholesterol. Terminal modifications can also be useful for cross-linking an RNA agent to another moiety. The skilled person knows that the mentioned modifications, such as PS, PS2, and/or VP may be combined, such that a molecule comprises at least two of the mentioned modifications and as disclosed in WO2018185241.

In one aspect of the invention, the therapeutic agent for use in treating an iron metabolism disease or condition further comprises a pharmaceutically-acceptable diluent, carrier, or excipient.

In certain aspects, the therapeutic agent of the invention is a pharmaceutical composition for use in the treatment of an iron metabolism disease or condition comprising an effective amount of the therapeutic agent of the invention and further comprising a pharmaceutically-acceptable diluent, carrier, or excipient.

In certain aspects, the pharmaceutical composition of the invention is adapted for delivery by a route selected from the group comprising: intravenous, intramuscular, subcutaneous, intraarticular, pulmonary, intranasal, intraocular, and intrathecal. In certain aspects, the pharmaceutical composition of the invention is adapted for delivery by subcutaneous route.

In certain aspects, the delivery route is selected from the group comprising: intravenous, intramuscular, subcutaneous, intraarticular, pulmonary, intranasal, intraocular, and intrathecal. In certain embodiments the delivery route is subcutaneous.

In one aspect of the invention, the iron metabolism disease or condition is a disease or condition that would further benefit from a cell explantation, implantation, or transplantation therapy, such as hematopoietic stem cell transplantation (HSCT).

In certain aspects, the treatment of the iron metabolism disease or condition further comprises the step of cell explantation, implantation, or transplantation therapy. In certain aspects, it comprises stem cell transplantation, preferably HSCT.

In certain aspects, the disease or condition treated using the compositions and methods of the present invention may be a non-malignant disease or condition. The non-malignant disease or condition is selected from the group consisting of severe aplastic anemia, hemoglobinopathies, like thalassemias and/or sickle cell disease, aplastic anemia, fanconi anemia, Wiskott Aldrich Syndrome, Hurlers Syndrome, familial haemophagocytic lymphohistiocytosis (FHL), chronic granulomatous disease (CGD), Kostmanns syndrome, Severe immunodeficiency syndrome, severe combined immune deficiency syndrome, or autoimmune disorders such as SLE, Multiple sclerosis, IBD, Crohn's Disease, Sjörgen's syndrome, vasculitis, Lupus, Myasthenia Gravis, Wegener's disease, malignant infantile osteopetrosis, mucoplysaccharidosis, paroxysmal nocturnal hemoglobinuria, pyruvate kinase deficiency, inborn errors of metabolisms, and/or other immunodeficiencies or autoimmune diseases.

In certain aspects, the disease or condition treated using the compositions and methods of the present invention may be a malignant disease or condition. The malignant disease or condition is selected from the group consisting of malignant disease or condition is selected from the group consisting of myelodysplastic syndromes (MDS), leukaemia (e.g., acute lymphoblastic leukemia (ALL), acute myelogenous leukemia (AML), acute monocytic leukemia (AMoL), chronic lymphocytic leukemia (CLL), chronic myelogenous leukemia (CML) and other leukemias (such as hairy cell leukemia (HCL), T-cell prolymphocytic leukemia (T-PLL), large granular lymphocytic leukemia and adult T-cell leukemia), lymphoma (e.g., Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, solid tumors (e.g. renal, hepatic and pancreatic cancer), Mycosis fungoides, Peripheral T-cell lymphoma not otherwise specified, Nodular sclerosis form of Hodgkin lymphoma, Mixed-cellularity subtype of Hodgkin lymphoma, multiple myeloma, neuroblastoma, Ewing sarcoma, and glioma.

In certain aspects, the object of the invention is to treat a hematologic disease or hematological malignancies. In certain aspects, the hematological malignancy is a leukemia such as ALL, AML, or AMoL. In certain aspects, the hematological disorder is MDS. In certain aspects, the disease or condition is CML, CLL, other leukemias and lymphoma. In certain aspects, the disease or condition to be treated is a hematological disease such as ALL, AML, AMoL, or MDS, requiring a stem cell transplantation, such as allogenic HSCT. In certain aspects, the disease or condition is AML. In certain aspects the disease or condition is MDS.

The most common type of transplantation of hematopoietic stem cells is derived usually from bone marrow, peripheral blood, or umbilical cord blood. Hematopoietic stem cells (HSCs) are pluripotent or multipotent cells that give rise to many or all blood cell types, i.e., the myeloid (monocytes and macrophages, neutrophils, basophils, eosinophils, erythrocytes, megakaryocytes/platelets, dendritic cells), and lymphoid lineages (T-cells, B-cells, NK-cells). It is known that a small number of HSCs can expand to generate a very large number of daughter HSCs. This phenomenon is used in hematopoietic stem cell transplantation (HSCT), when a relatively small number of HSCs reconstitute the hematopoietic system. The HSCT may be for example allogeneic (from another individual), or autologous (from the same individual).

In certain aspects of the invention, the HSCT is autologous, allogenic, syngeneic, or xenogeneic, gene modified, or gene edited.

In certain aspects, the HSCT is a first HSCT. In certain aspects, the HSCT is a retransplantation, e.g., after relapse.

Before transplantation, a conditioning for HSCT takes place, i.e., the recipient's immune system is usually destroyed with for example radiation or chemotherapy. This step is performed with the intention of eradicating the subject's malignant cell population and decreases the risk of rejection of the new immune HSCs at the cost of partial or complete bone marrow ablation, i.e., destruction of the patient's bone marrow ability to grow new blood cells. The stem cells to be transplanted are then transfused into the recipient subject's bloodstream where sufficient numbers find their way to the bone marrow space, where they replace the damaged hematopoietic system and resume the subject's normal blood cell production.

In one aspect of the invention, the therapeutic agent or pharmaceutical composition of the invention for use in the treatment of a mentioned disease or condition, further comprises co-administration of one or more chemotherapeutic agent, radiotherapy and/or immunotherapy.

In certain aspects, the one or more chemotherapeutic agent may be selected from the group consisting of busulfan, cyclophosphamide, fludarabine, treosulphane, melphalan, and thiotepa. In certain aspects, chemotherapy may be low intensity conditioning.

In certain aspects, the radiotherapy is selected from the group consisting of total body irradiation, total lymphoid irradiation, and total marrow irradiation.

In certain aspects, chemotherapy and radiation may be combined.

In certain aspects, the therapeutic agent or pharmaceutical composition of the invention is administered to the subject in need thereof prior to, during or after conditioning for HSCT. In certain aspects, the therapeutic agent or pharmaceutical composition of the invention is administered to the subject in need thereof prior to conditioning for HSCT.

In certain aspects, the therapeutic agent or pharmaceutical composition of the invention is administered to the subject in need thereof prior to, during or after receiving HSCT. In certain aspects, the therapeutic agent or pharmaceutical composition of the invention is administered to the subject in need thereof prior to receiving HSCT.

HSCT is a procedure associated with many potential complications, such as infection and graft-versus-host disease (GVHD), but as the treatment modality has improved and survival increased, its use has expanded beyond cancer, such as inborn errors of metabolism and autoimmune diseases. A transplant offers a chance for cure or long-term remission if complications such as GVHD, and the spectrum of opportunistic infections can be surmounted. Accordingly, an improved method of transplantation, which minimizes the risks and complications associated with the transplantation and which offer a more efficient transplantation is needed.

In one aspect of the invention, the therapeutic agent or the pharmaceutical composition of the invention improves at least one of HSCT efficacy, including improvement of engraftment, improvement of overall survival, reduction of non-relapse mortality, reduction of infections and idiopathic pneumonia syndrome, reduction of HSC-T related morbidities, like sinusoidal obstruction syndrome, chronic liver disease, GVHD, and/or GVL.

In certain aspects of the invention, the therapeutic agent or the pharmaceutical composition of the invention improves HSCT efficacy, including improvement of engraftment, improvement of overall survival, reduction of non-relapse mortality, reduction of infections and idiopathic pneumonia syndrome, reduction of HSC-T related morbidities, like sinusoidal obstruction syndrome, chronic liver disease, and GVHD.

In certain aspects, the therapeutic agent or the pharmaceutical composition of the invention is capable of preventing and/or reducing graft-versus-host disease (GVHD) and/or graft-versus-leukemia (GVL). In certain aspects it is capable of preventing and/or reducing graft-versus-host disease (GVHD).

The inventors have found that in the peritransplant setting, reduction of TMPRSS6 gene expression in the liver by treatment with TMPRSS6 siRNA will raise hepcidin levels in the circulation, reduce iron levels in the circulation, and reduce the generation of non-transferrin bound iron. By reducing iron mediated toxicity, this approach surprisingly enhances the engraftment of HSC donor cells, limits post-HSCT predisposition to infections, and reduces non relapse mortality.

In one aspect, the therapeutic agent for use or the pharmaceutical composition for use in the treatment of a mentioned disease or condition, comprises or consists of the step of:
(a) inhibiting TMPRSS6,
(b) blocking ferroportin, or
(c) increasing hepcidin,
   wherein the systemic iron level, NTBI, transferrin saturation, labile plasma iron, and/or eLPI level is decreased.

In one aspect, the invention relates to a method of treating an iron metabolism disease or condition in a subject in need thereof, wherein the treatment comprises administering an effective amount of a therapeutic agent or pharmaceutical composition of the invention to said subject. In certain aspects, the subject in need thereof is a subject in need of cell explant, implant, or transplant therapy. In certain aspects, the iron metabolism disease is associated with at least one of systemic iron level overload, transferrin saturation (Tsat), non-transferrin-bound iron (NTBI) overload, and labile plasma iron (LPI) overload.

In certain aspects, the treatment of the iron metabolism disease or condition is associated with a therapy involving cell explantation, implantation, or transplantation, such as HSCT.

In one aspect, the invention relates to a method of treating an iron metabolism disease or condition in a subject in need thereof, wherein the therapy comprises a therapy involving the step of cell explantation, implantation, or transplantation, such as HSCT.

In one aspect, the invention relates to the use of a therapeutic agent or a pharmaceutical composition according to the invention for the manufacture of a medicament for the treatment of an iron metabolism disease or condition.

In certain aspects, the treatment of the iron metabolism disease or condition further comprises a therapy involving cell explantation, implantation, or transplantation, such as HSCT.

In one aspect, the invention relates to the use of a therapeutic agent or a pharmaceutical composition according to the invention for the manufacture of a medicament for the treatment of an iron metabolism disease or condition, wherein the treatment comprises a therapy involving the step of cell explantation, implantation, or transplantation, such as HSCT.

As used herein, the term “bone marrow transplantation” or “stem cell transplantation” should be considered as interchangeable, referring to the transplantation of stem cells to a recipient. The stem cells do not necessarily have to be derived from bone marrow but could also be derived from other sources such as umbilical cord blood.

As used herein, the term “iron metabolism disease or condition” refers to a disease or condition that is associated to at least one of systemic iron overload, transferrin saturation, non-transferrin-bound iron (NTBI) overload, and labile plasma iron (LPI) overload. In particular, where a subject exhibits at least one of systemic iron overload, transferrin saturation, non-transferrin-bound iron (NTBI) overload, and labile plasma iron (LPI) overload.

As used herein, the term “HSCT” refers to a transplantation of hematopoietic stem cells to a recipient, wherein the stem cells usually are collected from bone marrow, peripheral blood, or umbilical cord blood.

As used herein, the term “prior to transplantation” in the context of administering a therapeutic agent or pharmaceutical composition of the invention, refers to a timeframe of hours, days or weeks before the transplantation. In particular, the therapeutic agent or pharmaceutical composition of the invention is administered a few weeks up to a few months, such as 1-10 weeks or 1-4 months before the conditioning for transplantation or before transplantation. The therapeutic agent or pharmaceutical composition may be administered continuously during this time period, or at a single, or few occasions. In certain aspects of the invention the therapeutic agent or pharmaceutical composition is administered in a single dose. The therapeutic agent or pharmaceutical composition should be administered in an effective dose, at a number of occasions and for a sufficient time before transplantation, so that a sufficient reduction of systemic iron level, Transferrin saturation (Tsat), non-transferrin-bound iron (NTBI), and/or labile plasma iron (LPI) as needed takes place. Such administration protocol is considered close at hand for the skilled person having access to the given therapeutic agent of pharmaceutical composition.

As used herein, the terms “subject” and “patient” are used interchangeably. As used herein, a subject is an animal, preferably a mammal such as a non-primate (e.g., cows, pigs, horses, cats, dogs, rats, etc.) and a primate (e.g., monkey (e.g., a rhesus monkey, a cynomolgus monkey or chimpanzee) and a human). In certain aspects of the invention, the subject is a human.

As used herein, the term “recipient” in the context of transplantation refers to the subject receiving the transplantation, in contrast to the “donor”, which is the subject the material (cells) to be transplanted originates from. In an allogeneic setting, the recipient and the donor are different individuals, in an autologous setting, the recipient and the donor is the same individual. In a syngeneic setting, the donor and recipient are different individuals but are genetically identical. Xenogeneic setting means the donor is from another species than the recipient.

As used herein, the terms “administering”, or “administration” refers to delivering of a therapeutic agent or pharmaceutical composition of the invention by any suitable method known in the art.

As used herein, the term “effective amount” refers to the amount of a therapy (e.g., a therapeutic agent) which is sufficient to reduce and/or ameliorate the severity and/or duration of a disease or condition, or a symptom thereof, prevent advancement of said disease or condition, cause regression, prevent recurrence, development, or onset of one or more symptoms associated with said disease or condition, or enhance or improve the prophylactic or therapeutic effect(s) of another therapy (e.g., another prophylactic or therapeutic agent). An effective amount of the therapeutic agent or pharmaceutical composition of the invention used prior, during or after HSCT, is thus the amount which is sufficient to result in the reduction of at least one of systemic iron level, transferrin saturation (Tsat), non-transferrin-bound iron (NTBI), and labile plasma iron (LPI), in such a way that it improves HSCT efficacy, including improvement of engraftment, improvement of overall survival, reduction of non-relapse mortality, reduction of infections and idiopathic pneumonia syndrome, reduction of HSC-T related morbidities, like sinusoidal obstruction syndrome, chronic liver disease, GVHD, and/or GVL.

As used herein, “an agent” is any purified or isolated natural or chemically synthesized entity comprising one or mole molecules.

In the method and use for manufacture of the therapeutic agent or pharmaceutical composition of the invention, a therapeutically effective amount of the active component is provided. A therapeutically effective amount can be determined by the ordinary skilled medical or veterinary worker based on patient characteristics, such as age, wight, sex, condition, complications, other diseases, etc., as is well known in the art.

TABLE 6
Summary abbreviations table

| Abbreviation | Meaning |
|---|---|
| mA, mU, mC, | 2′-O-Methyl RNA nucleotides |
| mG | |
| 2′-OMe | 2′-O-Methyl modification |
| fA, fU, fC, fG | 2′ deoxy-2′-F RNA nucleotides |
| 2′-F | 2′-fluoro modification |
| (ps) | phosphorothioate |
| (ps2) | phosphorodithioate |
| (vp) | Vinyl-(E)-phosphonate |
| (vp)-mU | 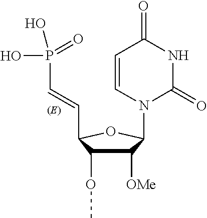 |
| (vp)-mU-phos | 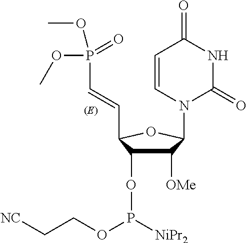 |
| ivA, ivC, ivU, | inverted RNA (3′-3′) nucleotides |
| ivG | |
| ST23 | 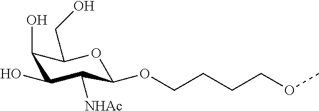 |
| ST23-phos | 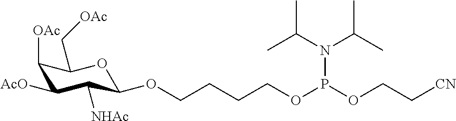 |
| ST43 (or C6XLT) | 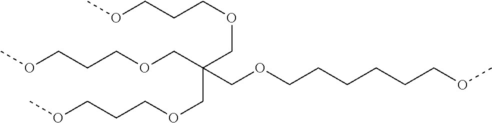 |
| ST43-phos (or C6XLT-phos) | 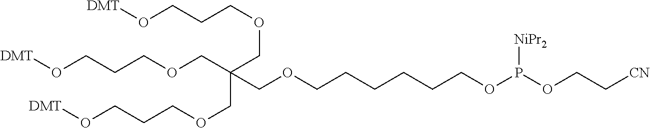 |
| Ser (GN) (when at the end of a chain, one of the O--- is OH) | 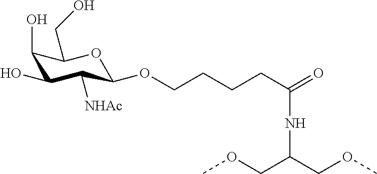 |
| [ST23(ps)]3 ST43(ps), or [ST23(ps)]3 C6XLT (ps) | 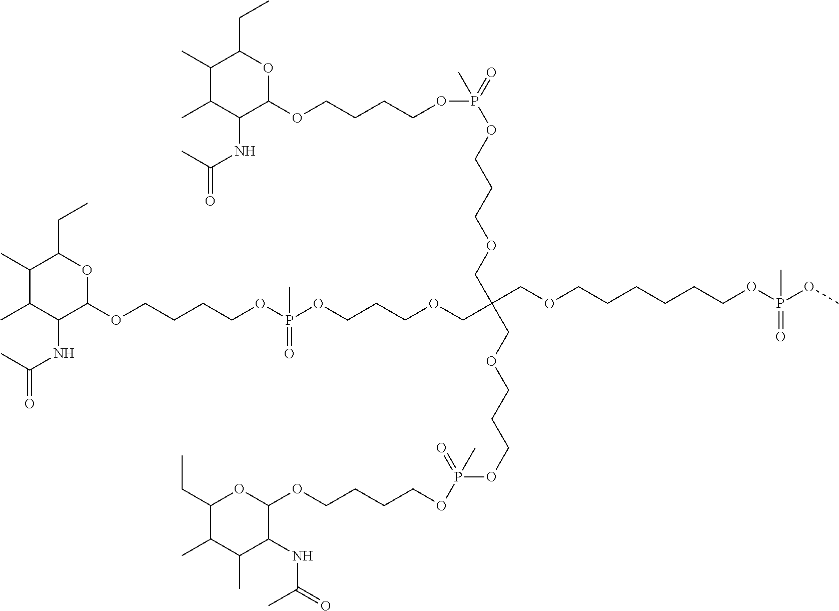 |
| [ST23]3 ST43 | 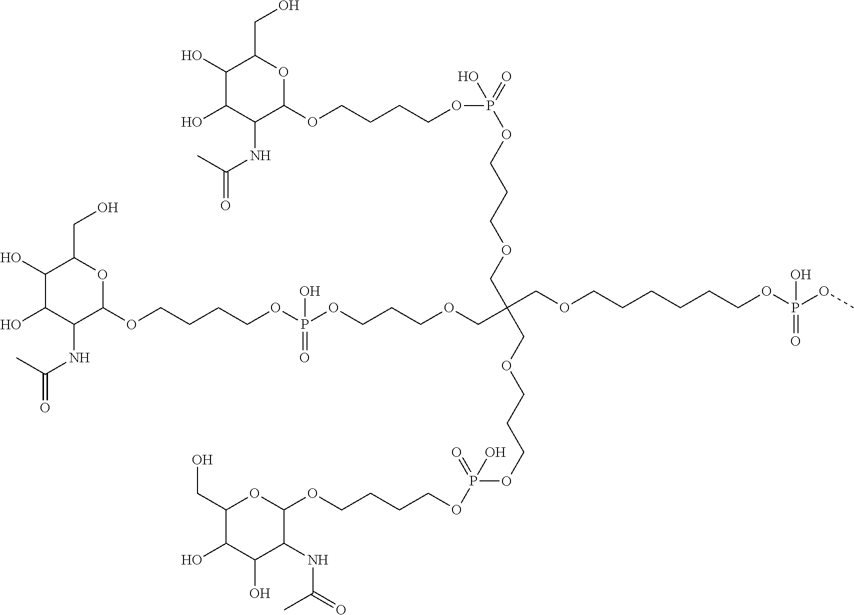 |
| [ST23(ps)]3 ST41(ps), or [ST23(ps)]3 C4XLT(ps) | 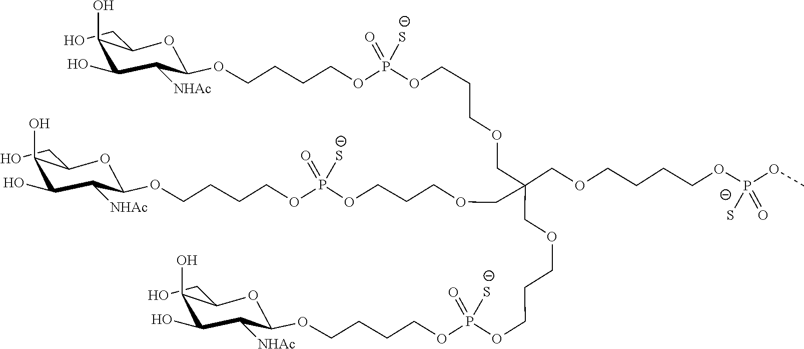 |
| [ST23]3 ST41 | 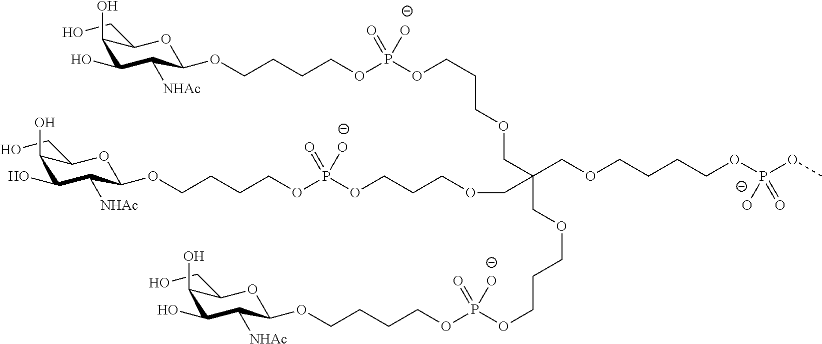 |

Preferred, non-limiting examples which embody certain aspects of the invention are described with reference to the following figures:

### BRIEF DESCRIPTION OF THE FIGURES

FIG. 1 Different treatment schedules of chemotherapeutic conditioning are depicted, which were evaluated in combination with application of siRNAs, like EU401 for impact on TMPRSS6 target gene expression, HAMP mRNA induction and systemic iron levels at Day +2. Depicted are the total doses of each, Busulfan and Fludarabine, that were administered at low-dose, mid-dose or high-dose chemotherapy. The low-dose consisted of a daily intraperitoneal (i.p.) injection of 0.8 mg/kg Fludarabine from Day −6 to Day −2, and a daily i.p. dose of 3.2 mg/kg Busulfan on Day −4 and Day −3. The mid-dose consisted of daily i.p. injection of 10 mg/kg Fludarabine from Day −6 to Day −2, and daily i.p. doses of 10 mg/kg Busulfan on Day −4 and Day −3. The high-dose consisted of daily i.p. injection of 10 mg/kg Fludarabine from Day −6 to Day −2, and twice daily i.p. doses of 16.25 mg/kg Busulfan on Day −4 and Day −3.

FIG. 2 Treatment with EU401 reduces TMPRSS6 mRNA and raises HAMP mRNA in liver of mice exposed to different doses of chemotherapeutic conditioning (low and high doses, see FIG. 1). Mean values+/−SD.

FIG. 3 Treatment with EU401 abrogates and attenuates the induction of serum iron, transferrin saturation, and NTBI in mice treated with a mid and a high dose of chemotherapeutic conditioning, respectively. Mean values+/−SEM.

FIG. 4 The impact by TMPRSS6 siRNA treatment on bone marrow transplant was evaluated in two different experimental conditions: A) Mice treated with the mid-dose of chemotherapeutic conditioning after receiving EU403 or EU402 siRNA at a dose of 5 mg/kg on Day −28 and Day −7. B) Mice treated with the high-dose of chemotherapeutic conditioning after receiving EU403 or EU402 siRNA at a dose of 5 mg/kg on Day −42, Day −21, and Day −1. BM donor cells were transplanted on Day 0 and chimerism was assessed as a measure of engraftment in the peripheral blood at 4-, 6-, and 8-weeks post-transplant and in tissues at 8 weeks post-transplant.

FIG. 5 The impact of TMPRSS6 siRNA treatment on post-transplant blood chimerism in the two different experimental conditions described in FIG. 4. The percentage of CD45.1⁺ donor-derived blood cells is shown at the respective time point of blood collection. Blood chimerism is superior at 4- and 8-weeks post-transplant in mice receiving mid-dose conditioning and EU402 treatment (A), and at 4-, 6-, and 8-weeks post-transplant in mice receiving high-dose conditioning and EU402 treatment (B). Data are presented are mean values+/−SEM.

FIG. 6 The impact of TMPRSS6 siRNA treatment on post-transplant blood myeloid and lymphoid engraftment in the two different experimental conditions described in FIG. 4. The percentage of CD45.1⁺ donor-derived CD11b⁺ CD3⁻ CD19⁻ myeloid and CD11b⁻ CD3⁺ CD19⁺ lymphoid cells is shown at the respective time point of blood collection. Blood myeloid and lymphoid chimerism is superior at 4- and 8-weeks post-transplant in mice receiving mid-dose conditioning and EU402 treatment (A), and at 4-, 6-, and 8-weeks post-transplant in mice receiving high-dose conditioning and EU402 treatment (B). Data are presented are mean values+/−SEM.

FIG. 7 The impact of TMPRSS6 siRNA treatment on post-transplant blood myeloid engraftment in the two different experimental conditions described in FIG. 4. The percentage of CD45.1⁺ donor-derived CD11b⁺ Ly6G⁻ L6C^{+/-} monocytes and CD11b⁺ Ly6G⁺ neutrophils is shown at the respective time point of blood collection. Blood myeloid chimerism is superior at 4- and 8-weeks post-transplant in mice receiving mid-dose conditioning and EU402 treatment (A), and at 4-, 6-, and 8-weeks post-transplant in mice receiving high-dose conditioning and EU402 treatment (B). Data are presented are mean values+/−SEM.

FIG. 8 The impact of TMPRSS6 siRNA treatment on post-transplant blood lymphoid engraftment in the two different experimental conditions described in FIG. 4. The percentage of CD45.1⁺ donor-derived CD11b⁻ CD19⁺ B-cells and CD11b⁻ CD3⁺ T-cells is shown at the respective time point of blood collection. Blood lymphoid chimerism is superior at 4- and 8-weeks post-transplant in mice receiving mid-dose conditioning and EU402 treatment (A), and at 4-, 6- and 8-weeks post-transplant in mice receiving high-dose conditioning and EU402 treatment (B). Data are presented as mean+/−SEM.

FIG. 9 The impact of TMPRSS6 siRNA treatment on post-transplant bone marrow and spleen chimerism in the two different experimental conditions described in FIG. 4. The percentage of CD45.1⁺ donor-derived cells in the bone marrow and spleen is shown at 8 weeks post-transplant. Bone marrow and spleen chimerism is superior in mice receiving mid-dose (A) and high-dose (B) conditioning and EU402 treatment at 8 weeks post-transplant. Data are presented as mean+/−SEM.

FIG. 10 The impact of TMPRSS6 siRNA treatment on post-transplant bone marrow myeloid and lymphoid engraftment in the two different experimental condition as described in FIG. 4: The percentage of CD45.1⁺ donor-derived CD11b⁺ myeloid and CD11b⁻ CD19⁺ CD3⁺ lymphoid cells in bone the marrow is shown at 8 weeks post-transplant. Bone marrow myeloid and lymphoid chimerism is superior in mice receiving mid-dose (A) and high-dose (B) conditioning and EU402 treatment at 8 weeks post-transplant. Data are presented as mean+/−SEM.

FIG. 11 The impact of TMPRSS6 siRNA treatment on post-transplant bone marrow multilineage engraftment in the two different experimental conditions described in FIG. 4. The percentage of CD45.1⁺ donor-derived CD45.1⁺ donor-derived CD11b⁺ Ly6G⁻ Ly6C^{+/-} monocytes, CD11b⁺ Ly6G⁺ neutrophils, CD11b⁻ CD19⁺ B-cells and CD11b⁻ CD3⁺ T-cells in the bone marrow is shown at 8 weeks post-transplant. Bone marrow multilineage chimerism is superior in mice receiving mid-dose (A) and high-dose (B) conditioning and EU402 treatment at 8 weeks post-transplant. Data are presented as mean+/−SEM.

FIG. 12 The impact of TMPRSS6 siRNA treatment on post-transplant splenic multilineage engraftment in the two different experimental conditions described in FIG. 4. The percentage of CD45.1⁺ donor-derived CD11b⁺ Ly6G⁻ Ly6C^{+/-} monocytes, CD11b⁺ Ly6G⁺ neutrophils, CD11b⁻ CD19⁺ B-cells and CD11b⁻ CD3⁺ T-cells in the spleen is shown at 8 weeks post-transplant. Splenic multilineage chimerism is superior in mice receiving mid-dose (A) and high-dose (B) conditioning and EU402 treatment at 8 weeks post-transplant. Data are presented as mean+/−SEM.

FIG. 13 The impact of TMPRSS6 siRNA treatment on of the number of hematopoietic stem and progenitor cells (HSPCs) in the two different experimental conditions described in FIG. 4. The percentage of bone marrow Lin⁻ Sca⁺ cKit⁺ (LSK) HSPCs over total Lin⁻ cells is shown at 8 weeks post-transplant. HSPC percentage remains unchanged in mice receiving mid-dose (A) and high-dose (B) conditioning and EU402 vs EU403 treatment at 8 weeks post-transplant. Data are presented as mean+/−SEM.

FIG. 14 The impact of TMPRSS6 siRNA treatment on bone marrow hematopoietic stem and progenitor cells (HSPCs) chimerism in the two different experimental conditions described in FIG. 4. The percentage of CD45.1⁺ donor-derived Lin⁻ Sca⁺ cKit⁺ (LSK) HSPCs and Lin-Sca1⁻ cKit⁺ myeloid progenitors (MPs) in the bone marrow is shown at 8 weeks post-transplant. LSK HSPC and MP chimerism is superior in mice receiving mid-dose (A) and high-dose (B) conditioning and EU402 treatment at 8 weeks post-transplant. Data are presented as mean+/−SEM.

FIG. 15 Experimental set up for testing the impact of TMPRSS6 siRNA treatment on bone marrow transplant outcome in murine model for MDS. NUP98-HOXD13 mice were treated with a dose of 5 mg/kg EU400 or EU401 on Day −35, Day −14 and Day 7 and received mid-dose chemotherapeutic conditioning. BM donor cells were transplanted on Day 0 and chimerism was assessed as a measure of engraftment in the peripheral blood at 3-, 8-, and 12-weeks post-transplant and in tissues at 12 weeks post-transplant.

FIG. 16 The impact of TMPRSS6 siRNA treatment on post-transplant blood engraftment in MDS mice receiving mid-dose conditioning. The percentage of CD45.1⁺ donor-derived blood cells (A) and CD45.1⁺ donor-derived CD11b⁺ Ly6G⁻ Ly6C^{+/-} monocytes and CD11b⁺ Ly6G⁺ neutrophils is shown at the respective time point of blood collection. Blood monocyte and neutrophils chimerism is superior at 3- and 8-weeks post-transplant in MDS mice receiving mid-dose conditioning and EU401 treatment (B). Data are presented are mean values+/−SEM.

FIG. 17 The impact of TMPRSS6 siRNA treatment on post-transplant bone marrow and spleen engraftment in MDS mice receiving mid-dose conditioning. The percentage of CD45.1⁺ donor-derived bone marrow and spleen cells (A) and CD45.1⁺ donor-derived CD11b⁺ myeloid cells and CD11b⁻ CD19⁺ CD3⁺ lymphocytes is shown at 12 weeks post-transplant. Bone marrow and spleen donor chimerism show a trend to be higher in MDS mice receiving mid-dose conditioning and EU401 treatment. Data are presented are mean values+/−SEM.

FIG. 18 Treatment with EU401 reduces the expression of inflammatory cytokines in bone marrow macrophages and neutrophils of transplanted MDS mice. Bone marrow samples were collected 12 weeks after transplant. A) Expression of the inflammatory cytokines IL1b, TNF-alpha and IFN-gamma in bone marrow CD11b^{low} F4/80⁺ macrophages (A) and CD11b⁺ Ly6G⁺ neutrophils (B) from transplanted MDS mice treated with EU400 or EU401 at 12 weeks post-transplant. Cytokines expressed by the respective cell populations are expressed as fold change over EU400-treated transplanted MDS mice. Mean values+/−SEM.

FIG. 19 Treatment of murine hepatocytes with EU401 reduces TMPRSS6 mRNA levels to a similar extent as EU402. Values obtained for TMPRSS6 mRNA were normalized to values generated for the house keeping gene, ACTIN, and related to mean of untreated sample (ut) set at 1-fold target gene expression. Each bar represents mean+/−SD from three biological replicates. Mean values+/−SD.

FIG. 20 Experimental set up for testing the impact of TMPRSS6 siRNA treatment on bone marrow transplant outcome in murine model for MDS. NUP98-HOXD13 mice were treated sc with a dose of 5 mg/kg EU403 or EU402 on Day −60, Day −30 and Day −1 and they received low-dose chemotherapeutic conditioning or high dose conditioning. Depicted are the total doses of each, Busulfan and Fludarabine, that were administered at A) low dose or B) high dose chemotherapy. The low dose consisted of a daily intraperitoneous (i.p.) injection of 10 mg/kg Fludarabine from Day −6 to Day −2, and twice a day i.p. doses of 2.5 mg/kg Busulfan on Day −4 and Day −3. The high dose consisted of daily i.p. injection of 10 mg/kg Fludarabine from Day −6 to Day −2, and twice a day i.p. doses of 16.25 mg/kg Busulfan on Day −4 and Day −3. The. BM donor cells were transplanted on Day 0 and chimerism was assessed as a measure of engraftment in the peripheral blood at 4, 6 and 8 weeks post-transplant and in tissues at 8 weeks post-transplant.

FIG. 21 The impact of TMPRSS6 siRNA treatment on post-transplant blood chimerism in MDS mice in the two different experimental conditions described in FIG. 20. The percentage of CD45.1⁺ donor-derived blood cells is shown at the respective time point of blood collection. Blood chimerism is superior at 4, 6 and 8 weeks post-transplant in mice receiving low-dose conditioning and EU402 treatment (A), and in mice receiving high-dose conditioning and EU402 treatment (B). Data are presented are mean values+/−SEM.

FIG. 22 The impact of TMPRSS6 siRNA treatment on post-transplant blood myeloid and lymphoid engraftment in the two different experimental conditions described in FIG. 4. The percentage of CD45.1⁺ donor-derived CD11b⁺ myeloid cells and CD11b⁻ lymphoid cells is shown at the respective time point of blood collection. Blood myeloid and lymphoid chimerism is superior at 4 and 8 weeks post-transplant in mice receiving mid-dose conditioning and EU402 treatment (A), and at 4, 6 and 8 weeks post-transplant in mice receiving high-dose conditioning and EU402 treatment (B). Data are presented are mean values+/−SEM.

FIG. 23 The impact of TMPRSS6 siRNA treatment on post-transplant blood myeloid engraftment in MDS mice in the two different experimental conditions described in FIG. 20. The percentage of CD45.1⁺ donor-derived CD11b⁺ Ly6G⁻ L6C^{+/-} monocytes and CD11b⁺ Ly6G⁺ neutrophils is shown at the respective time point of blood collection. Blood myeloid chimerism is superior at 6 and 8 weeks post-transplant in mice receiving low-dose and EU402 treatment (A), and or high-dose conditioning and EU402 treatment (B). Data are presented are mean values+/−SEM.

FIG. 24 The impact of TMPRSS6 siRNA treatment on post-transplant blood lymphoid engraftment in MDS mice in the two different experimental conditions described in FIG. 20. The percentage of CD45.1⁺ donor-derived CD11b⁻ CD19⁺ B-cells and CD11b⁻ CD3⁺ T-cells is shown at the respective time point of blood collection. Blood T lymphoid chimerism is superior at 4, 6 and 8 weeks post-transplant in mice receiving low-dose conditioning and EU402 treatment (A), or high-dose conditioning and EU402 treatment (B). Data are presented as mean+/−SEM.

FIG. 25 The impact of TMPRSS6 siRNA treatment on post-transplant bone marrow chimerism in the two different experimental conditions described in FIG. 20. At 8 weeks post-transplant bone marrow chimerism is superior in MDS mice receiving EU402 treatment at both, either low-dose (A, C, E) or high-dose (B, D, F) conditioning. Data are presented as mean+/−SEM. The percentage of total CD45.1⁺ donor-derived cells in the bone marrow and the percentage of CD45.1⁺ donor-derived CD11b⁺ myeloid and CD11b⁻ CD19⁺ CD3⁺ lymphoid cells in bone the marrow is shown in A) and B). The percentage of donor-derived CD45.1⁺, CD11b⁺ Ly6G⁻ Ly6C^{+/-} monocytes, CD11b⁺ Ly6G⁺ neutrophils, CD11b⁻ CD19⁺ B-cells and CD11b⁻ CD3⁺ T-cells in the bone marrow is shown at 8 weeks post-transplant in C) and D). The percentage of CD45.1⁺ donor-derived Lin⁻ Sca⁺ cKit⁺ (LSK) HSPCs and Lin⁻ Sca1⁻ cKit⁺ myeloid progenitors (MPs) in the bone marrow is shown at 8 weeks post-transplant in E) and F).

### EXAMPLES

### Example 1—Inhibition of TMPRSS6 Expression Increases Hepcidin Levels by TMPRSS6 siRNA Treatment in Rodent Model for Chemotherapeutic Conditioning and Stem Cell Transplantation

This example shows the inhibition of TMPRSS6 expression and the induction of hepcidin expression by EU401 in mice treated with chemotherapeutic conditioning.

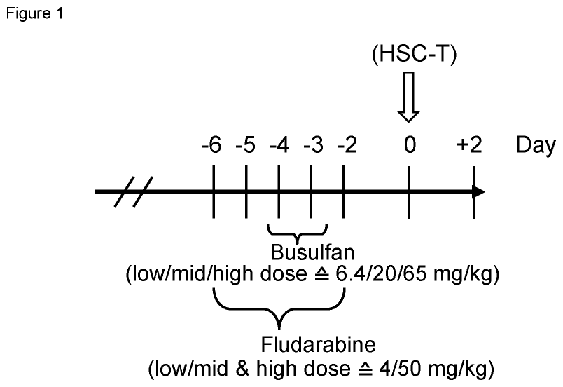

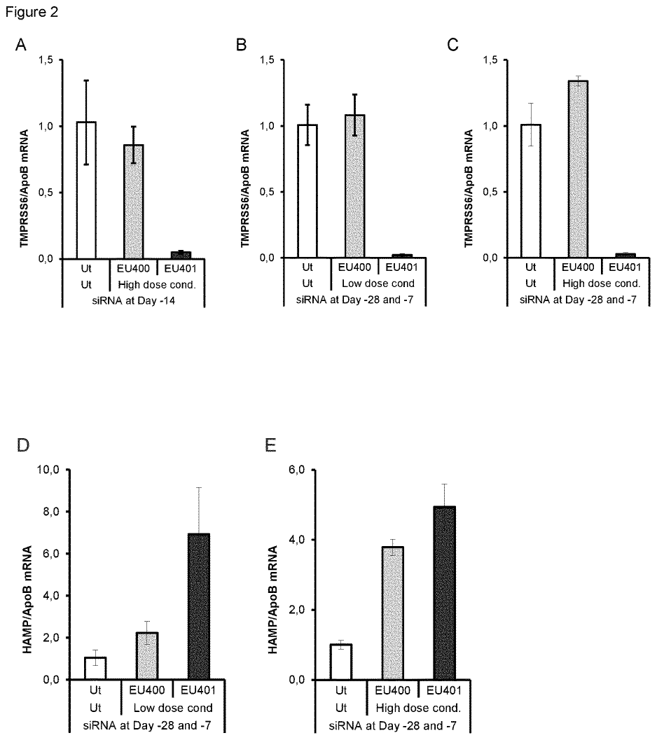

Wild-type mice were treated with either a low dose or high dose of chemotherapeutic conditioning (Day −6 to Day −2) as described above and depicted in FIG. 1. Prior to conditioning, mice received one dose of 5 mg/kg EU400 or EU401 on Day −14 by subcutaneous injection or two doses of either 5 mg/kg of EU400 or 5 mg/kg of EU401 on Day −28 and Day-7 via the same route. Liver tissue samples were collected on Day +2 for extraction of total RNA and assessment of target gene expression by qRT-PCR. Treatment with a single dose of EU401 (5 mg/kg) on Day −14 reduced hepatic TMPRSS6 mRNA expression in mice that had received a high dose of chemotherapeutic conditioning (FIG. 2A). Similarly, two doses of EU401 lowered TMPRSS6 expression in mice that had received either the low dose or high dose of chemotherapeutic conditioning (FIGS. 2B and C). Two doses of EU401 were also effective to induce hepatic Hamp expression in mice that received either the low dose or high dose chemotherapeutic conditioning (FIGS. 1D and E). The high dose of conditioning led to significant elevation of hepatic HAMP expression compared to untreated mice, which was further increased by treatment with EU401. The siRNA molecules used in this study are depicted in Table 5. Experimental set up is shown in FIG. 1 and results are shown in FIG. 2.

### Example 2—TMPRSS6 siRNA Treatment Decreases Serum Iron, Transferrin Saturation, and NTBI in Rodent Model for Chemotherapeutic Conditioning and Stem Cell Transplantation

The example shows that EU401 treatment attenuates the increase of serum iron levels, transferrin saturation (Tsat) and non-transferrin bound iron (NTBI) induced by a mid- or high-dose of chemotherapeutic conditioning.

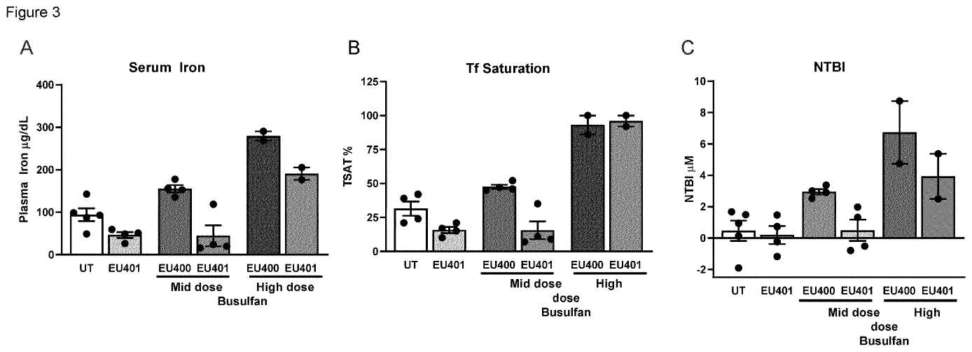

Wild-type mice were either non-treated (UT) or received subcutaneous administration of 5 mg/kg EU401 on Day −28 and Day −7, alone or followed by a mid-dose or high-dose of chemotherapeutic conditioning (Day −6 to Day −2) as depicted in FIG. 1. Blood samples were collected on Day +2 for assessment of serum iron, transferrin saturation (Tsat) and non-transferrin bound iron (NTBI). Treatment with EU401 alone lowered serum iron levels and transferrin saturation in otherwise untreated wild-type mice. In mice that had received a mid-dose or high-dose of chemotherapeutic conditioning, treatment with siRNA EU401 lowered serum iron levels Tsat and NTBI compared to mice that had received the non-targeting siRNA control, EU400 (FIG. 3). The suppression of NTBI was complete in mice that had received the mid-dose of chemotherapy conditioning and partial in those that received the high-dose of chemotherapy conditioning. The siRNA molecules used in this study are depicted in Table 5. Experimental set up is shown in FIG. 1 and results are shown in FIG. 3.

### Example 3—Impact of TMPRSS6 siRNA Treatment on the Outcome of Bone Marrow Transplant in Myelosuppressed Wild-Type Mice

This example shows that the treatment of recipient mice with TMPRSS6 siRNA EU401 enhances engraftment and donor chimerism after bone marrow transplant.

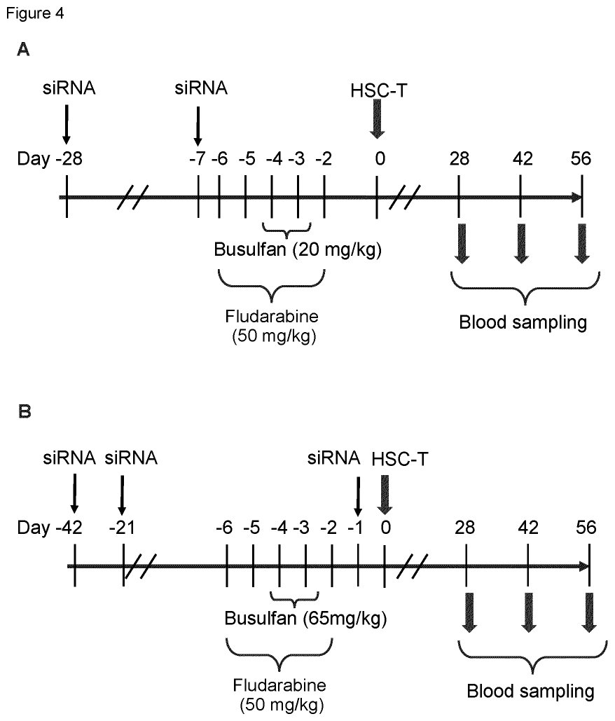

To investigate the impact of TMPRSS6 siRNA treatment on the outcome of bone marrow transplant, we used the murine models of chemotherapeutic conditioning described earlier (see FIG. 1) and performed bone marrow cell transplantation (see treatment scheme in FIGS. 4A and B). CD45.2⁺ wild-type recipient mice were first treated by a mid-dose or high-dose of chemotherapeutic conditioning and then on Day 0 received 1×10⁷ donor cells from CD45.1⁺ wild-type donors by IV injection into the tail vein.

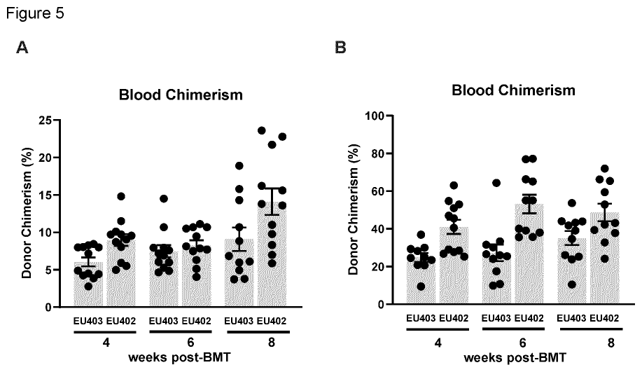

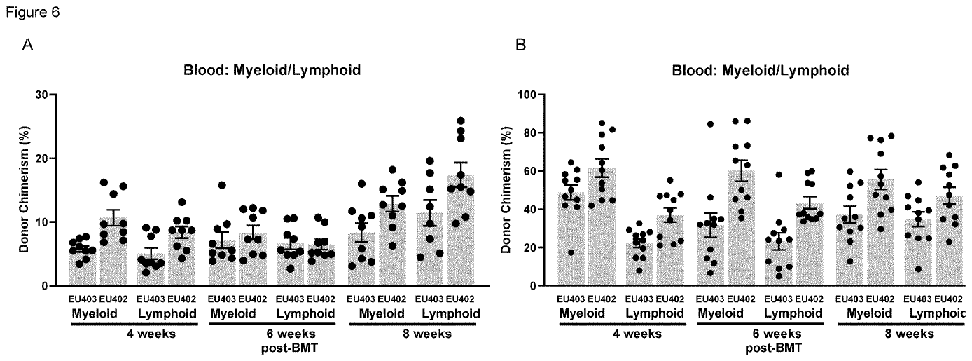

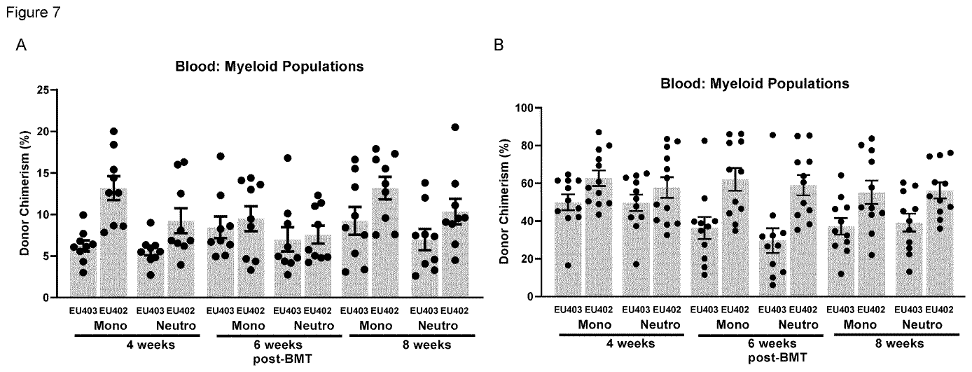

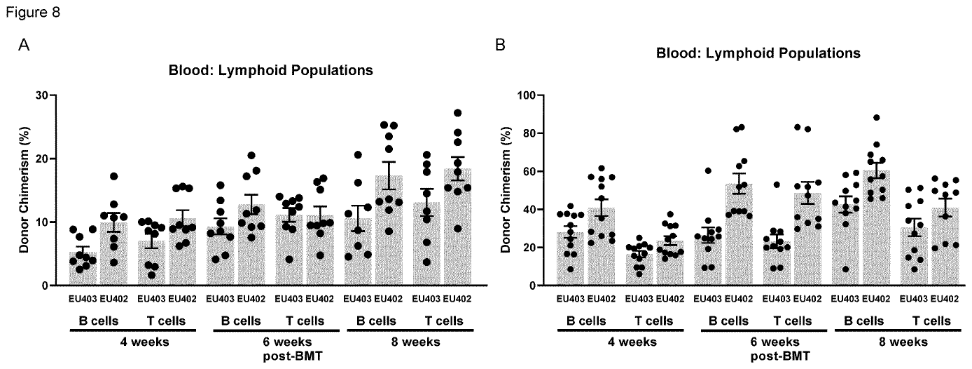

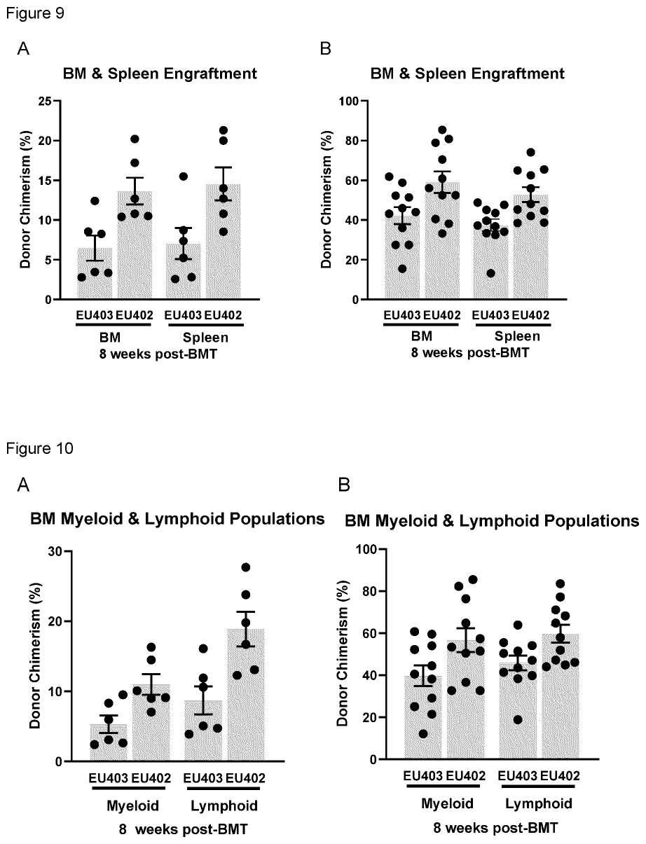

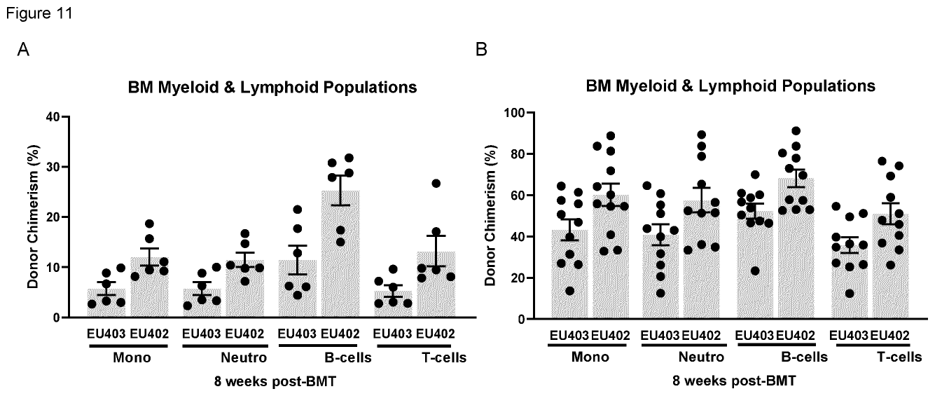

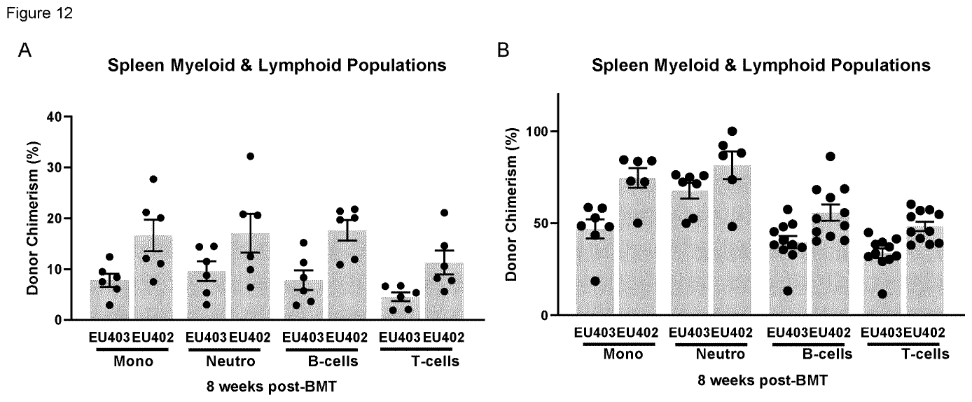

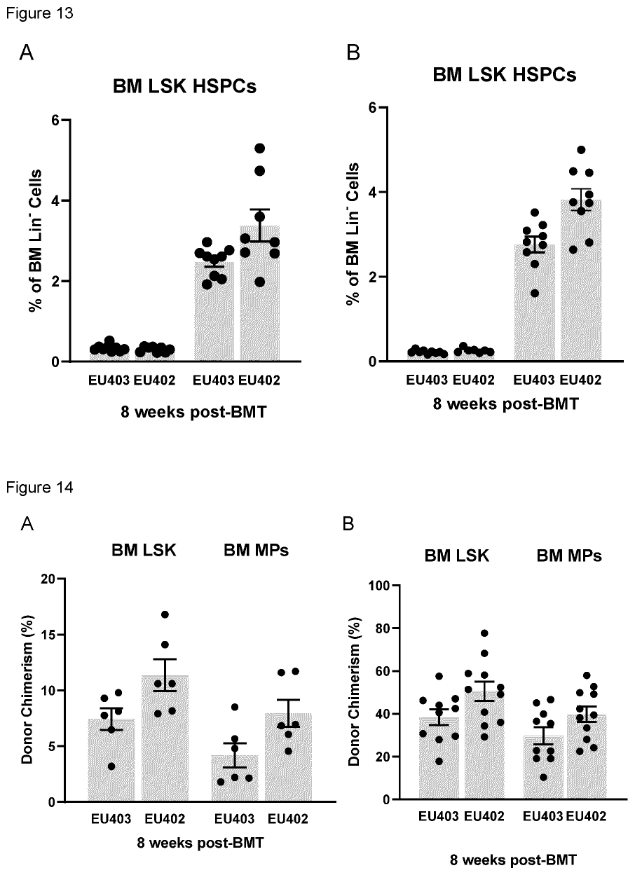

The impact of TMPRSS6 inhibition on engraftment was assessed by treating the mice also with TMPRSS6 siRNA EU402 and by comparison to treatment with a non-targeting control siRNA, EU403. Mice receiving the mid dose of chemotherapeutic conditioning were treated with 5 mg/kg respective siRNA on Day −28 and Day −7, whereas those receiving the high dose chemotherapy received 5 mg/kg siRNA on Day −42, Day −21, and Day −1. CD45.1+ chimerism as a measure of engraftment was assessed in blood samples collected at 4, 6, and 8 weeks after transplantation by flow cytometry using appropriate markers. Engraftment was higher in mice that had received the high dose of chemotherapy compared to mid-dose conditioning. Treatment with the TMPRSS6 siRNA EU402 enhanced donor cell engraftment following both mid- and high-dose conditioning. The siRNA molecules used in this study are depicted in Table 5. The experimental set up is depicted in FIG. 4 and results are shown in FIGS. 5 to 14.

### Example 4—Impact of TMPRSS6 siRNA Treatment on the Outcome of Bone Marrow Transplant in Mouse Model of Myelodysplastic Syndrome

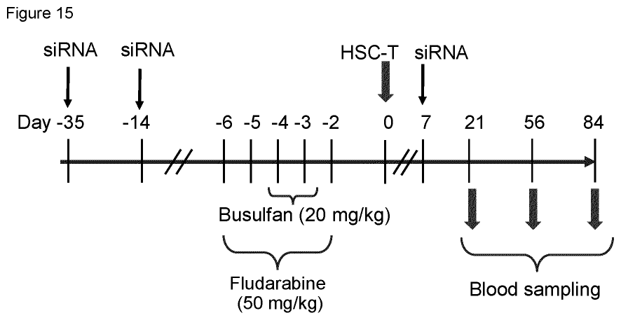

This example shows that treatment with TMPRSS6 siRNA enhances engraftment and donor chimerism in a mouse model for myelodysplastic syndrome. The impact of TMPRSS6 siRNA treatment on post-transplant engraftment was also tested in NUP98-HOXD13 transgenic mice, a murine model of myelodysplastic syndrome (MDS) (Lin et al., Blood 2005 Jul. 1; 106(1): 287-295). MDS mice were treated as depicted in FIG. 15 below. They were treated with 5 mg/kg EU401 or EU400 on Day −35, Day-14, and Day 7 by subcutaneous administration and received the mid-dose of chemotherapy conditioning. 1×10⁷ bone marrow cells from CD45.1⁺ donor mice were transplanted on Day 0 in CD45.2⁺ MDS mice and blood samples were collected at weeks 3, 8, and 12 thereafter.

The example shows that treatment with EU401 improves bone marrow transplant outcome in MDS mice treated with mid-dose conditioning. This effect is observed by a trend to increased chimerism in the CD45.1⁺ donor-derived blood populations as well as in the myeloid population at 3 and 8 weeks after transplantation. Similarly, the proportion of donor derived cells shows a trend to be higher in the bone marrow and spleen of MDS mice treated with EU401 compared to EU400 at the end of the study (week 12 after the transplantation).

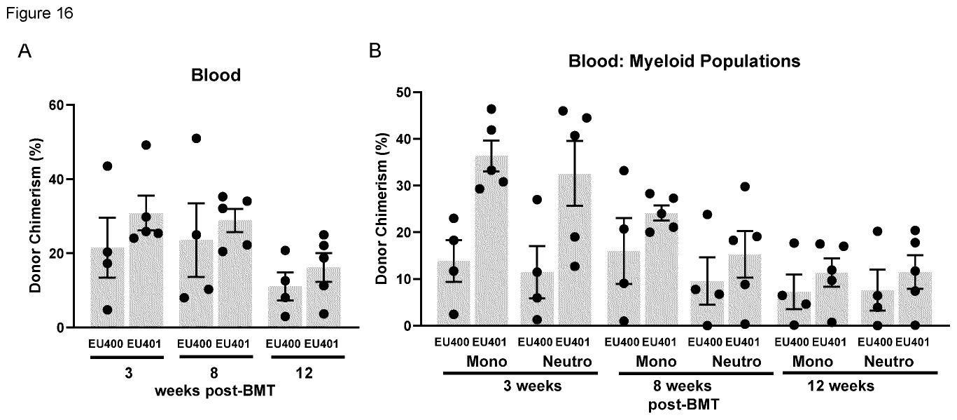

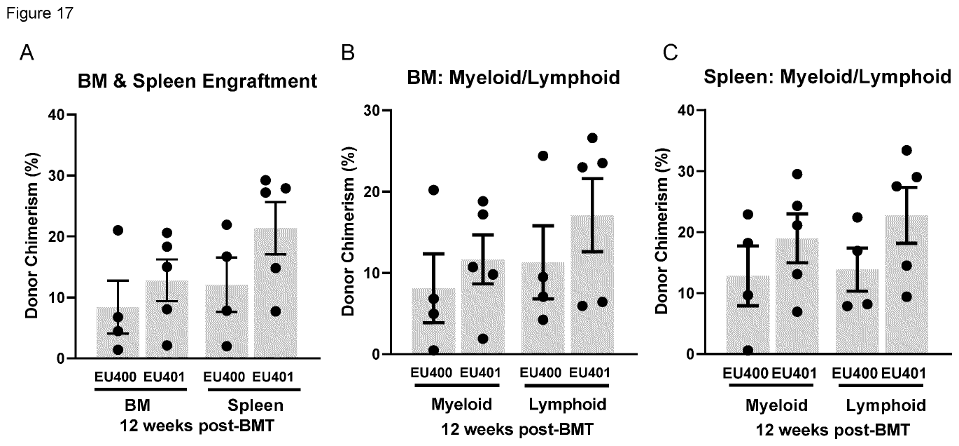

The siRNA molecules used in this study are depicted in Table 5. Experimental set up is shown in FIG. 15 and results are shown in FIGS. 16 and 17.

### Example 5—Reduction of Inflammatory Cytokines by TMPRSS6 in the Bone Marrow

This example shows that treatment with TMPRSS6 siRNA lowers the post-transplant expression of inflammatory cytokines in an animal model of myelodysplastic syndrome.

The impact of TMPRSS6 siRNA treatment on post-transplant inflammatory cytokine production was assessed in NUP98-HOXD13 transgenic mice, a murine model for myelodysplastic syndrome (Blood 2005 Jul. 1; 106(1): 287-295).

MDS mice were treated as depicted in FIG. 15 below. They were treated with 5 mg/kg EU401 or EU400 on Day −35, Day −14, and Day −7 by subcutaneous administration and received the mid-dose of chemotherapy conditioning. 1×10⁷ bone marrow cells from CD45.1⁺ donor mice were transplanted on Day 0 in CD45.2⁺ MDS mice and blood samples were collected at weeks 3, 8, and 12 thereafter.

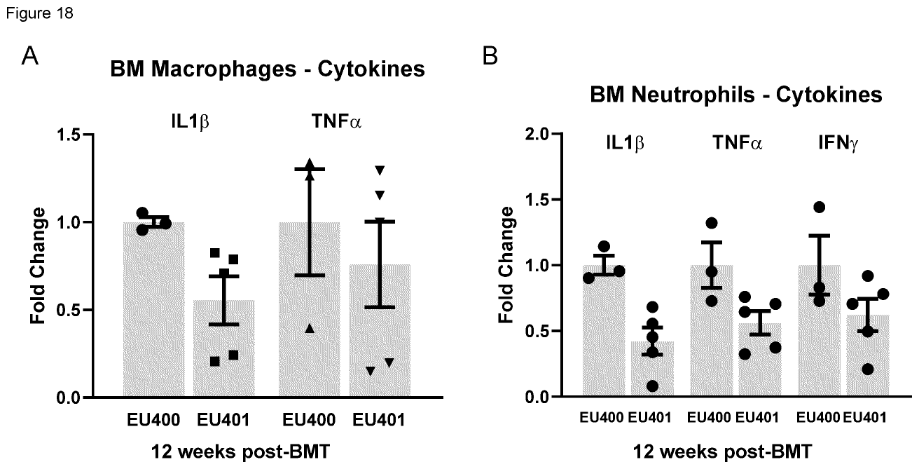

The expression of cytokines was assessed by flow cytometry in bone marrow CD11b^{low} F4/80⁺ macrophages and CD11b⁺ Ly6G⁺ neutrophils. The siRNA molecules used in this study are depicted in Table 5. Experimental set up is shown in FIG. 15 and results are shown in FIG. 18.

### Example 6—Reduction of TMPRSS6 mRNA Levels in Primary Hepatocytes

This example shows dose dependent reduction of TMPRSS6 mRNA levels in primary hepatocytes by EU401 and EU402 following receptor mediated uptake.

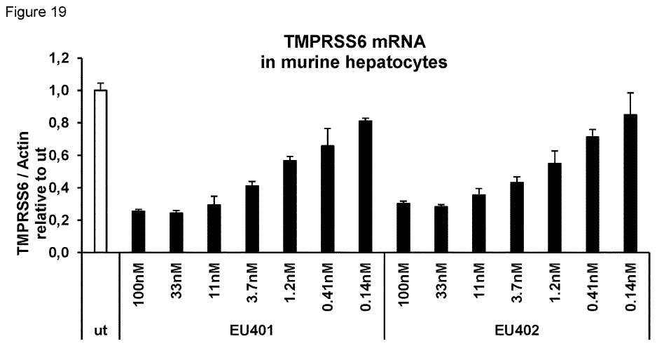

Primary mouse hepatocytes were seeded in a 96-well plate at a density of 25,000 cells per well. Cells were then incubated with the TMPRSS6 siRNA conjugates in the cell culture medium at 100 nM, 33 nM, 11 nM, 3.7, 1.2 nM, 0.41 nM, and 0.14 nM as indicated below. The following day, cells were lysed for RNA extraction and TMPRSS6 and Actin mRNA levels were determined by Taqman qRT-PCR. Values obtained for TMPRSS6 mRNA were normalized to values generated for the house-keeping gene, Actin, and related to the mean of untreated sample (ut) set at 1-fold target gene expression. Each bar represents mean+/−SD from three biological replicates. The siRNA conjugates used in this study are listed in Table 5. Experimental set up is shown in FIG. 15 and Results are shown in FIG. 19.

### Example 7—Impact of TMPRSS6 siRNA Treatment on the Outcome of Bone Marrow Transplant in Mouse Model of Myelodysplastic Syndrome

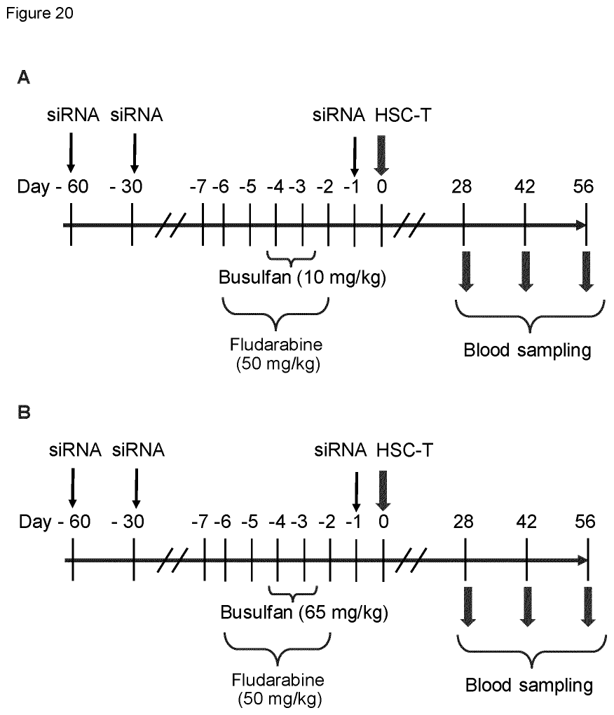

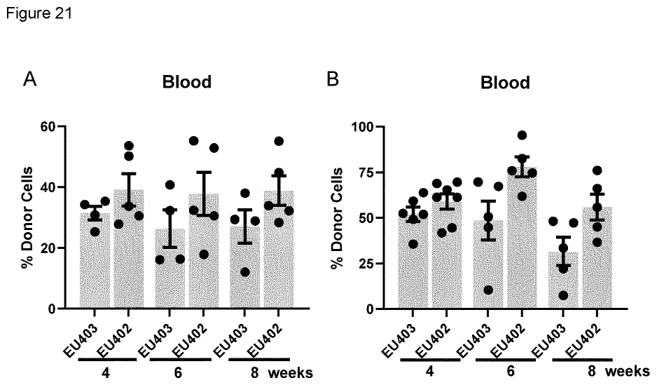

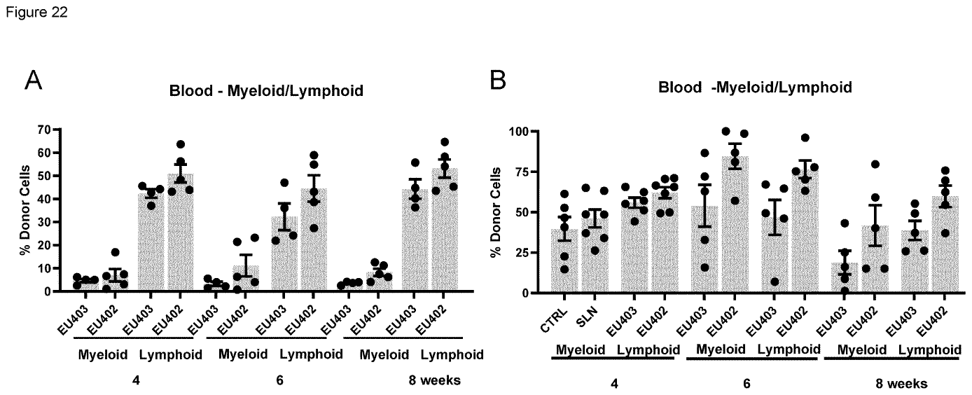

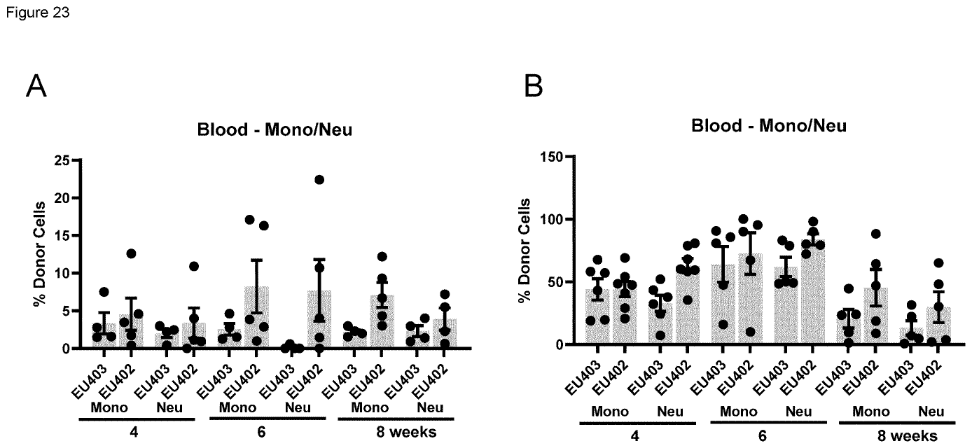

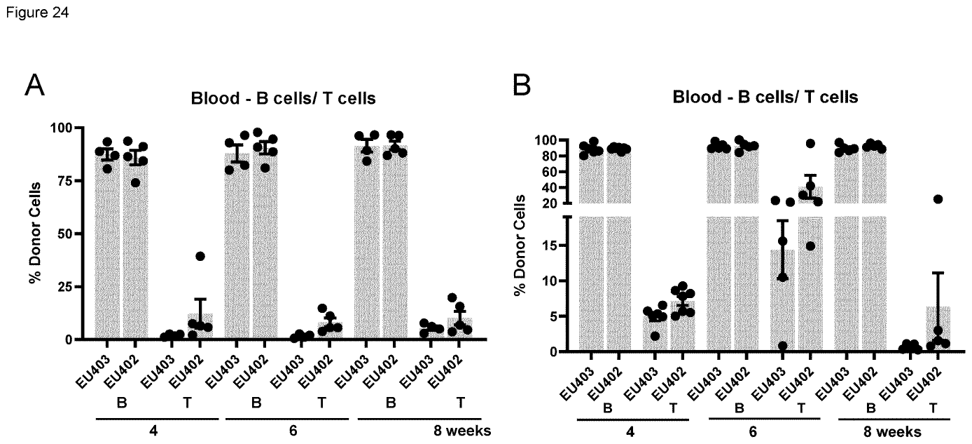

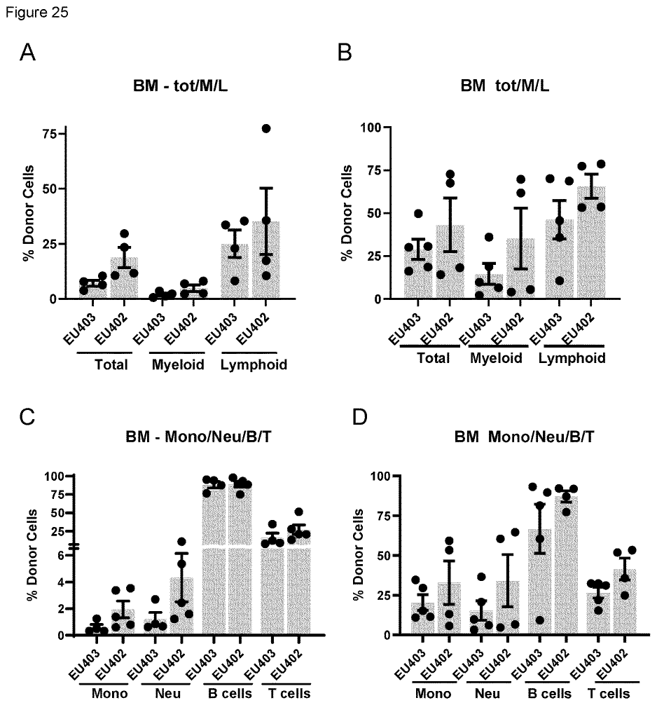

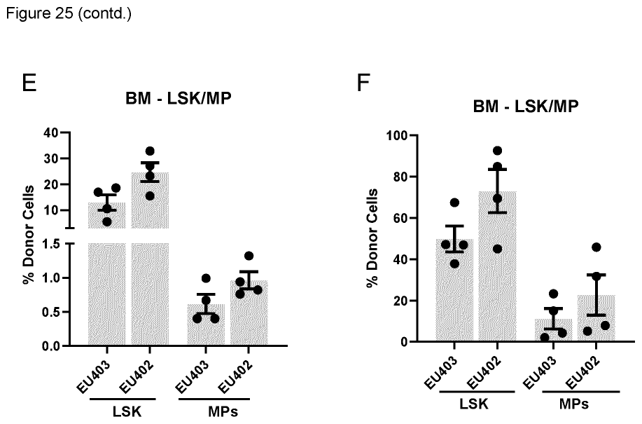

The example shows that the treatment with TMPRSS6 siRNA enhances engraftment and donor chimerism in a mouse model for myelodysplastic syndrome at a low and at a high dose of chemotherapeutic conditioning. The impact of TMPRSS6 siRNA treatment on post-transplant engraftment was tested in NUP98-HOXD13 transgenic mice, a murine model of myelodysplastic syndrome (MDS) (Lin et al., Blood 2005 Jul. 1; 106(1): 287-295). MDS mice were treated as depicted in FIG. 20 below. They were treated with 5 mg/kg EU403 n or EU402 on Day −60, Day −30 and Day −1 by subcutaneous administration and received the low or the high dose of chemotherapy conditioning as depicted in FIG. 20. 1×10⁷ bone marrow cells from CD45.1⁺ donor mice were transplanted on Day 0 in CD45.2⁺ MDS mice and blood samples were collected at week 4, 6 and 8 thereafter. CD45.1⁺ chimerism as a measure of engraftment was assessed in blood samples collected at 4, 6 and 8 weeks after transplantation by flow cytometry using appropriate markers. Engraftment was higher in mice that had received the high dose of chemotherapy compared to low dose conditioning, as expected. Treatment with the TMPRSS6 siRNA EU402 enhanced donor cell engraftment following both low and high dose conditioning. siRNA molecules used in this study are depicted in Table 1. The experimental set up is depicted in FIG. 20. Results are shown in FIG. 21 to 25.

### Statements

1. A therapeutic agent for use in the treatment of an iron metabolism disease or condition wherein the therapeutic agent comprises an inhibitor selected from:
(d) a TMPRSS6 inhibitor;
(e) a MT2 inhibitor; or
(f) a Ferroportin inhibitor,
and wherein the treatment comprises cell implantation, explantation, or transplantation.

2. A therapeutic agent for use according to statement 1, wherein the therapeutic agent is selected from:
(i) an antibody or antigen-binding fragment thereof, or a variant, fusion, or derivative of said antibody or antigen-binding fragment, or a fusion of said variant or derivative thereof;
(ii) antibody mimic (for example, based on non-antibody scaffolds); (hepcidin mimetic) selected from the group consisting of affibody, tetranectin, adnectin (monobody), anticalin, DARPin (ankyrin), avimer, iMab, microbody, peptide aptamer, Kunitz domain, and affilin
(iii) RNA aptamer;
(iv) small molecule;
(v) CovX-body; or
(vi) nucleic acid;
wherein (i) or (ii) may be selected from the group consisting of affibody, tetranectin, adnectin (monobody), anticalin, DARPin (ankyrin), avimer, iMab, microbody, peptide aptamer, Kunitz domain, and affilin, and any combination thereof.

3. A therapeutic agent for use according to statement 2, wherein the nucleic acid comprises at least one duplex region that comprises at least a portion of a first strand and at least a portion of a second strand that is at least partially complementary to the portion of the first strand, wherein said first strand is at least partially complementary to at least a portion of RNA transcribed from the TMPRSS6 gene and is capable of inhibiting expression of TMPRSS6.

4. A therapeutic agent for use according to any one of statements 2 or 3, wherein one or more nucleotides on the first and/or second strand are modified, to form modified nucleotides.

5. A therapeutic agent for use according to any one of statements 2 to 4, wherein the nucleic acid is conjugated to a ligand, optionally at the 5′ end of the second strand.

6. A therapeutic agent for use according to statements 5, wherein the ligand comprises (i) one or more N-acetyl galactosamine (GalNAc) moieties and derivatives thereof, and (ii) a linker, wherein the linker conjugates the GalNAc moieties to the nucleic acid.

7. A therapeutic agent for use according to any one of statements 5 or 6, wherein the linker is a bivalent, trivalent, or tetravalent branched structure.

8. A therapeutic agent for use according to any one of statements 2 to 7, wherein the nucleic acid is any of the mentioned in Tables 1, 2, 3, 4, or 5.

9. The therapeutic agent for use according to any one of statements 1 to 8, wherein the nucleic acid is EU401 or EU402.

10. A pharmaceutical composition for use in the treatment of an iron metabolism disease or condition, the pharmaceutical composition comprising an effective amount of the therapeutic agent as defined in any of the preceding claims, further comprising a pharmaceutically acceptable diluent, carrier, or excipient.

11. The pharmaceutical composition for use of statements 10, adapted for delivery by a route selected from the group comprising: intravenous, intramuscular, subcutaneous, intraarticular, pulmonary, intranasal, intraocular, and intrathecal.

12. The pharmaceutical composition for use of statements 10-11, wherein the delivery route is selected from the group comprising: intravenous, intramuscular, subcutaneous, intraarticular, pulmonary, intranasal, intraocular, and intrathecal.

13. The therapeutic agent for use or the pharmaceutical composition for use of any of the preceding statements, wherein the disease or condition is
a) non-malignant, wherein the non-malignant disease or condition is selected from the group consisting of severe aplastic anemia, hemoglobinopathies, like thalassemias and/or sickle cell disease, aplastic anemia, fanconi anemia, Wiskott Aldrich Syndrome, Hurlers Syndrome, familial haemophagocytic lymphohistiocytosis (FHL), chronic granulomatous disease (CGD), Kostmanns syndrome, Severe immunodeficiency syndrome, severe combined immune deficiency syndrome, or autoimmune disorders such as SLE, Multiple sclerosis, IBD, Crohn's Disease, Sjörgen's syndrome, vasculitis, Lupus, Myasthenia Gravis, Wegener's disease, malignant infantile osteopetrosis, mucoplysaccharidosis, paroxysmal nocturnal hemoglobinuria, pyruvate kinase deficiency, inborn errors of metabolisms, and/or other immunodeficiencies or autoimmune diseases;
   or
b) wherein the disease is malignant, wherein the malignant disease or condition is selected from the group consisting of myelodysplastic syndromes (MDS), leukaemia (e.g., acute lymphoblastic leukemia (ALL), acute myelogenous leukemia (AML), acute monocytic leukemia (AMoL), chronic lymphocytic leukemia (CLL), chronic myelogenous leukemia (CML) and other leukemias (such as hairy cell leukemia (HCL), T-cell prolymphocytic leukemia (T-PLL), large granular lymphocytic leukemia and adult T-cell leukemia), lymphoma (e.g., Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, solid tumors (e.g. renal, hepatic and pancreatic cancer), Mycosis fungoides, Peripheral T-cell lymphoma not otherwise specified, Nodular sclerosis form of Hodgkin lymphoma, Mixed-cellularity subtype of Hodgkin lymphoma, multiple myeloma, neuroblastoma, Ewing sarcoma, and glioma.

14. A therapeutic agent for use or the pharmaceutical composition for use of any of the preceding statements, wherein the treatment comprises or consists of the step of:
(a) inhibiting TMPRSS6,
(b) inhibiting MT2,
(c) blocking ferroportin, or
(c) increasing hepcidin
wherein the systemic iron level, NTBI, transferrin saturation, labile plasma iron, and/or eLPI level are decreased.

15. A therapeutic agent for use or the pharmaceutical composition for use of any of the preceding statements, wherein the treatment further comprises co-administration of one or more chemotherapeutic agent, radiotherapy, and/or immunotherapy.

16. A therapeutic agent for use or the pharmaceutical composition for use of any of the preceding statements, wherein the cell explantation, implantation, or transplantation treatment comprises hematopoietic stem cell transplantation (HSCT), optionally wherein the therapeutic agent or the pharmaceutical composition is administered to the subject in need thereof prior to, during, or after conditioning for HSCT,
or
wherein the agent is administered to the subject in need thereof prior to, during, or after receiving HSCT.

17. A therapeutic agent for use or the pharmaceutical composition for use of statements 16, wherein the agent is capable of preventing and/or reducing graft-versus-host disease (GVHD) and/or graft-versus-leukemia (GVL), and optionally further improves HSCT efficacy, including improvement of engraftment, improvement of overall survival, reduction of non-relapse mortality, reduction of infections and idiopapathic pneumonia syndrome, and/or reduction of HSC-T related morbidities, like sinusoidal obstruction syndrome, and chronic liver disease.

18. A method of treating an iron metabolism disease or condition in a subject in need thereof, comprising administering an effective amount of the pharmaceutical composition or the therapeutic agent defined in any of the preceding claims, wherein the treatment comprises cell implantation, explantation, or transplantation.

19. The method of statement 18, wherein the agent is selected from:
(i) an antibody or antigen-binding fragment thereof, or a variant, fusion, or derivative of said antibody or antigen-binding fragment, or a fusion of said variant or derivative thereof;
(ii) antibody mimic (for example, based on non-antibody scaffolds); (hepcidin mimetic) selected from the group consisting of affibody, tetranectin, adnectin (monobody), anticalin, DARPin (ankyrin), avimer, iMab, microbody, peptide aptamer, Kunitz domain, and affilin
(iii) RNA aptamer;
(iv) small molecule;
(v) CovX-body; or
(vi) nucleic acid;
wherein (i) or (ii) may be selected from the group consisting of affibody, tetranectin, adnectin (monobody), anticalin, DARPin (ankyrin), avimer, iMab, microbody, peptide aptamer, Kunitz domain, and affilin, and any combination thereof.

20. The method of statement 19, wherein the nucleic acid comprises at least one duplex region that comprises at least a portion of a first strand and at least a portion of a second strand that is at least partially complementary to the portion of the first strand, wherein said first strand is at least partially complementary to at least a portion of RNA transcribed from the TMPRSS6 gene and is capable of inhibiting expression of TMPRSS6.

21. The method of statement 20, wherein one or more nucleotides on the first and/or second strand are modified, to form modified nucleotides.

22. The method of statement 20, wherein the nucleic acid is conjugated to a ligand, optionally at the 5′ end of the second strand.

23. The method of statement 22, wherein the ligand comprises (i) one or more N-acetyl galactosamine (GalNAc) moieties and derivatives thereof, and (ii) a linker, wherein the linker conjugates the GalNAc moieties to the nucleic acid.

24. The method of statement 23, wherein the linker is a bivalent, trivalent or tetravalent branched structure.

25. The method of statement 20, wherein the nucleic acid is any of the mentioned in Tables 1, 2, 3, 4 or 5, preferably, wherein the nucleic acid is EU401 or EU402.

26. The method of statement 19, wherein the pharmaceutical composition comprises an effective amount of the therapeutic agent as defined in any of the preceding statements, further comprising a pharmaceutically acceptable diluent, carrier, or excipient.

27. The method of statement 26, wherein the pharmaceutical composition is adapted for delivery by a route selected from the group comprising: intravenous, intramuscular, subcutaneous, intraarticular, pulmonary, intranasal, intraocular, and intrathecal.

28. The method of statement 26, wherein the delivery route of the pharmaceutical composition is selected from the group comprising: intravenous, intramuscular, subcutaneous, intraarticular, pulmonary, intranasal, intraocular, and intrathecal.

29. The method of statement 18, wherein the disease or condition is
a) non-malignant, wherein the non-malignant disease or condition is selected from the group consisting of severe aplastic anemia, hemoglobinopathies, like thalassemias and/or sickle cell disease, aplastic anemia, fanconi anemia, Wiskott Aldrich Syndrome, Hurlers Syndrome, familial haemophagocytic lymphohistiocytosis (FHL), chronic granulomatous disease (CGD), Kostmanns syndrome, Severe immunodeficiency syndrome, severe combined immune deficiency syndrome, or autoimmune disorders such as SLE, Multiple sclerosis, IBD, Crohn's Disease, Sjörgen's syndrome, vasculitis, Lupus, Myasthenia Gravis, Wegener's disease, malignant infantile osteopetrosis, mucoplysaccharidosis, paroxysmal nocturnal hemoglobinuria, pyruvate kinase deficiency, inborn errors of metabolisms, and/or other immunodeficiencies or autoimmune diseases;
   or
b) wherein the disease is malignant, wherein the malignant disease or condition is selected from the group consisting of myelodysplastic syndromes (MDS), leukaemia (e.g., acute lymphoblastic leukemia (ALL), acute myelogenous leukemia (AML), acute monocytic leukemia (AMoL), chronic lymphocytic leukemia (CLL), chronic myelogenous leukemia (CML) and other leukemias (such as hairy cell leukemia (HCL), T-cell prolymphocytic leukemia (T-PLL), large granular lymphocytic leukemia and adult T-cell leukemia), lymphoma (e.g., Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, solid tumors (e.g. renal, hepatic and pancreatic cancer), Mycosis fungoides, Peripheral T-cell lymphoma not otherwise specified, Nodular sclerosis form of Hodgkin lymphoma, Mixed-cellularity subtype of Hodgkin lymphoma, multiple myeloma, neuroblastoma, Ewing sarcoma, and glioma.

30. The method of statement 18, wherein the treatment comprises or consists of the step of:
(a) inhibiting TMPRSS6,
(b) inhibiting MT2,
(c) blocking ferroportin, or
(c) increasing hepcidin
wherein the systemic iron level, NTBI, transferrin saturation, labile plasma iron, and/or eLPI level are decreased.

31. The method of statement 18, wherein the treatment further comprises co-administration of one or more chemotherapeutic agent, radiotherapy, and/or immunotherapy.

32. The method of statement 18, wherein the cell explant or transplant treatment comprises hematopoietic stem cell transplantation (HSCT), optionally wherein the therapeutic agent or the pharmaceutical composition is administered to the subject in need thereof prior to, during, or after conditioning for HSCT,
or
wherein the agent is administered to the subject in need thereof prior to, during, or after receiving HSCT.

33. The method of statement 32, wherein the agent is capable of preventing and/or reducing graft-versus-host disease (GVHD) and/or graft-versus-leukemia (GVL), and optionally further improves HSCT efficacy, including improvement of engraftment, improvement of overall survival, reduction of non-relapse mortality, reduction of infections and idiopathic pneumonia syndrome, and/or reduction of HSC-T related morbidities, like sinusoidal obstruction syndrome, and chronic liver disease.

34. Use of a therapeutic agent or a pharmaceutical composition according to the preceding claims for the manufacture of a medicament for the treatment of an iron metabolism disease or condition, wherein the treatment comprises cell explant or transplant.

35. The use of statement 34, wherein the agent is selected from:
(i) an antibody or antigen-binding fragment thereof, or a variant, fusion, or derivative of said antibody or antigen-binding fragment, or a fusion of said variant or derivative thereof;
(ii) antibody mimic (for example, based on non-antibody scaffolds); (hepcidin mimetic) selected from the group consisting of affibody, tetranectin, adnectin (monobody), anticalin, DARPin (ankyrin), avimer, iMab, microbody, peptide aptamer, Kunitz domain and affilin
(iii) RNA aptamer;
(iv) small molecule;
(v) CovX-body; or
(vi) nucleic acid;
wherein (i) or (ii) may be selected from the group consisting of affibody, tetranectin, adnectin (monobody), anticalin, DARPin (ankyrin), avimer, iMab, microbody, peptide aptamer, Kunitz domain and affilin, and any combination thereof.

36. The use of statement 35, wherein the nucleic acid comprises at least one duplex region that comprises at least a portion of a first strand and at least a portion of a second strand that is at least partially complementary to the portion of the first strand, wherein said first strand is at least partially complementary to at least a portion of RNA transcribed from the TMPRSS6 gene and is capable of inhibiting expression of TMPRSS6.

37. The use of statement 36, wherein one or more nucleotides on the first and/or second strand are modified, to form modified nucleotides.

38. The use of statement 36, wherein the nucleic acid is conjugated to a ligand, optionally at the 5′ end of the second strand.

39. The use of statement 38, wherein the ligand comprises (i) one or more N-acetyl galactosamine (GalNAc) moieties and derivatives thereof, and (ii) a linker, wherein the linker conjugates the GalNAc moieties to the nucleic acid.

40. The use of statement 39, wherein the linker is a bivalent, trivalent, or tetravalent branched structure.

41. The use of statement 36, wherein the nucleic acid is any of the mentioned in Tables 1, 2, 3, 4, or 5, preferably wherein the nucleic acid is EU401 or EU402.

42. The use of statement 36, wherein the pharmaceutical composition comprises an effective amount of the therapeutic agent as defined in any of the preceding statements, further comprising a pharmaceutically acceptable diluent, carrier, or excipient.

43. The use of statement 36, wherein the pharmaceutical composition is adapted for delivery by a route selected from the group comprising: intravenous, intramuscular, subcutaneous, intraarticular, pulmonary, intranasal, intraocular, and intrathecal.

44. The use of statement 36, wherein the delivery route of the pharmaceutical composition is selected from the group comprising: intravenous, intramuscular, subcutaneous, intraarticular, pulmonary, intranasal, intraocular, and intrathecal.

45. The use of statement 36, wherein the disease or condition is
a) non-malignant, wherein the non-malignant disease or condition is selected from the group consisting of severe aplastic anemia, hemoglobinopathies, like thalassemias and/or sickle cell disease, aplastic anemia, fanconi anemia, Wiskott Aldrich Syndrome, Hurlers Syndrome, familial haemophagocytic lymphohistiocytosis (FHL), chronic granulomatous disease (CGD), Kostmanns syndrome, Severe immunodeficiency syndrome, severe combined immune deficiency syndrome, or autoimmune disorders such as SLE, Multiple sclerosis, IBD, Crohn's Disease, Sjörgen's syndrome, vasculitis, Lupus, Myasthenia Gravis, Wegener's disease, malignant infantile osteopetrosis, mucoplysaccharidosis, paroxysmal nocturnal hemoglobinuria, pyruvate kinase deficiency, inborn errors of metabolisms, and/or other immunodeficiencies or autoimmune diseases;
   or
b) wherein the disease is malignant, wherein the malignant disease or condition is selected from the group consisting of myelodysplastic syndromes (MDS), leukaemia (e.g., acute lymphoblastic leukemia (ALL), acute myelogenous leukemia (AML), acute monocytic leukemia (AMoL), chronic lymphocytic leukemia (CLL), chronic myelogenous leukemia (CML) and other leukemias (such as hairy cell leukemia (HCL), T-cell prolymphocytic leukemia (T-PLL), large granular lymphocytic leukemia and adult T-cell leukemia), lymphoma (e.g., Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, solid tumors (e.g. renal, hepatic and pancreatic cancer), Mycosis fungoides, Peripheral T-cell lymphoma not otherwise specified, Nodular sclerosis form of Hodgkin lymphoma, Mixed-cellularity subtype of Hodgkin lymphoma, multiple myeloma, neuroblastoma, Ewing sarcoma, and glioma.

46. The use of statement 36, wherein the treatment comprises or consists of the step of:
(a) inhibiting TMPRSS6,
(b) inhibiting MT2,
(c) blocking ferroportin, or
(c) increasing hepcidin
wherein the systemic iron level, NTBI, transferrin saturation, labile plasma iron, and/or eLPI level are decreased.

47. The use of statement 36, wherein the treatment further comprises co-administration of one or more chemotherapeutic agent, radiotherapy, and/or immunotherapy.

48. The use of statement 36, wherein the cell explant or transplant treatment comprises hematopoietic stem cell transplantation (HSCT), optionally wherein the therapeutic agent or the pharmaceutical composition is administered to the subject in need thereof prior to, during, or after conditioning for HSCT,
or
wherein the agent is administered to the subject in need thereof prior to, during, or after receiving HSCT.

49. The use of statement 36, wherein the agent is capable of preventing and/or reducing graft-versus-host disease (GVHD) and/or graft-versus-leukemia (GVL), and optionally further improves HSCT efficacy, including improvement of engraftment, improvement of overall survival, reduction of non-relapse mortality, reduction of infections and idiopathic pneumonia syndrome, and/or reduction of HSC-T related morbidities, like sinusoidal obstruction syndrome, and chronic liver disease.

1. A therapeutic agent for use in the treatment of an iron metabolism disease or condition wherein the therapeutic agent comprises an inhibitor selected from:
(g) a TMPRSS6 inhibitor;
(h) a MT2 inhibitor; or
(i) a Ferroportin inhibitor,
and wherein the treatment comprises cell implantation, explantation, or transplantation.

2. A therapeutic agent for use according to claim 1, wherein the agent is selected from:
(i) an antibody or antigen-binding fragment thereof, or a variant, fusion, or derivative of said antibody or antigen-binding fragment, or a fusion of said variant or derivative thereof;
(ii) antibody mimic (for example, based on non-antibody scaffolds); (hepcidin mimetic) selected from the group consisting of affibody, tetranectin, adnectin (monobody), anticalin, DARPin (ankyrin), avimer, iMab, microbody, peptide aptamer, Kunitz domain, and affilin
(iii) RNA aptamer;
(iv) small molecule;
(v) CovX-body; or
(vi) nucleic acid;
wherein (i) or (ii) may be selected from the group consisting of affibody, tetranectin, adnectin, monobody, anticalin, DARPin, ankyrin, avimer, iMab, microbody, peptide aptamer, Kunitz domain, affilin, and any combination thereof.

3. A therapeutic agent for use according to claim 2, wherein the nucleic acid comprises at least one duplex region that comprises at least a portion of a first strand and at least a portion of a second strand that is at least partially complementary to the portion of the first strand, wherein said first strand is at least partially complementary to at least a portion of RNA transcribed from the TMPRSS6 gene and is capable of inhibiting expression of TMPRSS6.

4. A therapeutic agent for use according to any one of claims 2 or 3, wherein one or more nucleotides on the first and/or second strand are modified, to form modified nucleotides.

5. A therapeutic agent for use according to any one of claims 2 to 4, wherein the nucleic acid is conjugated to a ligand, optionally at the 5′ end of the second strand.

6. A therapeutic agent for use according to claim 5, wherein the ligand comprises (i) one or more N-acetyl galactosamine (GalNAc) moieties and derivatives thereof, and (ii) a linker, wherein the linker conjugates the GalNAc moieties to the nucleic acid.

7. A therapeutic agent for use according to any one of claims 5 or 6, wherein the linker is a bivalent, trivalent, or tetravalent branched structure.

8. A therapeutic agent for use according to any one of claims 2 to 7, wherein the nucleic acid is any of the mentioned in Tables 1, 2, 3, 4, or 5.

9. The therapeutic agent for use according to any one of statements 1 to 8, wherein the nucleic acid is EU401 or EU402.

10. A pharmaceutical composition for use in the treatment of an iron metabolism disease or condition, the pharmaceutical composition comprising an effective amount of the therapeutic agent as defined in any of the preceding claims, further comprising a pharmaceutically acceptable diluent, carrier, or excipient.

11. The pharmaceutical composition for use of claim 10, adapted for delivery by a route selected from the group comprising: intravenous, intramuscular, subcutaneous, intraarticular, pulmonary, intranasal, intraocular, and intrathecal.

12. The pharmaceutical composition for use of claims 10-11, wherein the delivery route is selected from the group comprising: intravenous, intramuscular, subcutaneous, intraarticular, pulmonary, intranasal, intraocular, and intrathecal.

13. The therapeutic agent for use or the pharmaceutical composition for use of any of the preceding claims, wherein the disease or condition is
a) non-malignant, wherein the non-malignant disease or condition is selected from the group consisting of severe aplastic anemia, hemoglobinopathies, like thalassemias and/or sickle cell disease, aplastic anemia, fanconi anemia, Wiskott Aldrich Syndrome, Hurlers Syndrome, familial haemophagocytic lymphohistiocytosis (FHL), chronic granulomatous disease (CGD), Kostmanns syndrome, Severe immunodeficiency syndrome, severe combined immune deficiency syndrome, or autoimmune disorders such as SLE, Multiple sclerosis, IBD, Crohn's Disease, Sjörgen's syndrome, vasculitis, Lupus, Myasthenia Gravis, Wegener's disease, malignant infantile osteopetrosis, mucoplysaccharidosis, paroxysmal nocturnal hemoglobinuria, pyruvate kinase deficiency, inborn errors of metabolisms, and/or other immunodeficiencies or autoimmune diseases;
or
b) wherein the disease is malignant, wherein the malignant disease or condition is selected from the group consisting of myelodysplastic syndromes (MDS), leukaemia (e.g., acute lymphoblastic leukemia (ALL), acute myelogenous leukemia (AML), acute monocytic leukemia (AMoL), chronic lymphocytic leukemia (CLL), chronic myelogenous leukemia (CML) and other leukemias (such as hairy cell leukemia (HCL), T-cell prolymphocytic leukemia (T-PLL), large granular lymphocytic leukemia and adult T-cell leukemia), lymphoma (e.g., Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, solid tumors (e.g. renal, hepatic and pancreatic cancer), Mycosis fungoides, Peripheral T-cell lymphoma not otherwise specified, Nodular sclerosis form of Hodgkin lymphoma, Mixed-cellularity subtype of Hodgkin lymphoma, multiple myeloma, neuroblastoma, Ewing sarcoma, and glioma.

14. A therapeutic agent for use or the pharmaceutical composition for use of any of the preceding claims, wherein the treatment comprises or consists of the step of:
(a) inhibiting TMPRSS6,
(b) inhibiting MT2,
(c) blocking ferroportin, or
(c) increasing hepcidin
wherein the systemic iron level, NTBI, transferrin saturation, labile plasma iron, and/or eLPI level are decreased.

15. A therapeutic agent for use or the pharmaceutical composition for use of any of the preceding claims, wherein the treatment further comprises co-administration of one or more chemotherapeutic agent, radiotherapy, and/or immunotherapy.

16. A therapeutic agent for use or the pharmaceutical composition for use of any of the preceding claims, wherein the cell explantation, implantation, or transplantation treatment comprises hematopoietic stem cell transplantation (HSCT), optionally wherein the therapeutic agent or the pharmaceutical composition is administered to the subject in need thereof prior to, during, or after conditioning for HSCT,
or
wherein the agent is administered to the subject in need thereof prior to, during, or after receiving HSCT.

17. A therapeutic agent or the pharmaceutical composition for use of claim 16, wherein the agent is capable of preventing and/or reducing graft-versus-host disease (GVHD) and/or graft-versus-leukemia (GVL), and optionally further improves HSCT efficacy, including improvement of engraftment, improvement of overall survival, reduction of non-relapse mortality, reduction of infections and idiopathic pneumonia syndrome, and/or reduction of HSC-T related morbidities, like sinusoidal obstruction syndrome, and chronic liver disease.

18. A method of treating an iron metabolism disease or condition in a subject in need thereof, comprising administering an effective amount of the pharmaceutical composition or the therapeutic agent defined in any of the preceding claims, wherein the treatment comprises cell explantation, implantation, or transplantation.

19. Use of a therapeutic agent or a pharmaceutical composition according to the preceding claims for the manufacture of a medicament for the treatment of an iron metabolism disease or condition, wherein the treatment comprises cell explantation, implantation, or transplantation.
